(19) United States
(12) Patent Application Publication
(10) Pub. No.: US 2006/0154894 A1
(43) Pub. Date: Jul. 13, 2006

(54) Biologically active surfaces and methods of their use

(75) Inventors: David A. Berry, Brookline, MA (US); Alireza Khademhosseini, Cambridge, MA (US); Kahp Y. Suh, Seoul (KR); Ram Sasisekharan, Bedford, MA (US); Robert S. Langer, Newton, MA (US)

Correspondence Address: WOLF GREENFIELD & SACKS, PC;NULL, FEDERAL RESERVE PLAZA, 600 ATLANTIC AVENUE, BOSTON, MA, 02210-2206, US

(73) Assignee: Massachusetts Institute of Technology, Cambridge, MA (US)

(21) Appl. No.: 11/229,488

(22) Filed: Sep. 15, 2005

Related U.S. Application Data
(60) Provisional application No. 60/610,361, filed on Sep. 15, 2004.

(51) Int. Cl. A61K 31/737; A61K 31/727

(52) U.S. Cl. 514/54; 514/56

(57) ABSTRACT

The invention relates to the immobilization of polysaccharides on a substrate. In particular, the invention relates to biologically active surfaces formed by the immobilization of glycosaminoglycans on a substrate. The invention also provides biologically active surfaces that contain one or more different glycosaminoglycans and, optionally, one or more other agents. These agents can be biological or therapeutic agents. The invention also relates to methods of using the surfaces of the invention, such as, methods of affecting biological processes, eliciting patterns of cellular response, screening, treatment, diagnosis and preventing food contamination and/or spoilage.

### RELATED APPLICATIONS

This application claims priority under 35 U.S.C. §119 from U.S. provisional application Ser. No. 60/610,361, filed Sep. 15, 2004. The entire contents of which is herein incorporated by reference.

### GOVERNMENT SUPPORT

Aspects of the invention may have been made using funding from National Institutes of Health grants EB-00244 and CA-52857 and US Army Research grant DAAD-19-02-D0002. Accordingly, the Government may have rights in the invention.

### FIELD OF THE INVENTION

The invention relates to the immobilization of polysaccharides on a substrate. In particular, the invention relates to biologically active surfaces formed by the immobilization of glycosaminoglycans on a substrate. The invention also provides biologically active surfaces that contain one or more different glycosaminoglycans and, optionally, one or more other agents. These agents can be biological or therapeutic agents. The invention also relates to methods of using the surfaces of the invention, such as, methods of affecting biological processes, eliciting patterns of cellular response, screening, treatment, diagnosis and preventing food contamination and/or spoilage.

### BACKGROUND

The formation of stable polysaccharide coatings has potential applications. To generate hyaluronic acid (HA)-coated surfaces various immobilization techniques have been employed ranging from covalent attachment, layer-by-layer deposition and binding with natural ligands such as p32. These strategies, however, involve approaches that require the use of chemicals, UV light or cumbersome procedures.

### SUMMARY OF THE INVENTION

This invention relates, in part, to substrates with polysaccharides immobilized thereon and methods of their use. The substrates with polysaccharides immobilized thereon are, preferably, biologically active surfaces. These biologically active surfaces can be or form part of filtering devices, medical devices, pills, particles, food storage devices, etc. The biologically active surfaces provided can be used in a variety of methods such as methods for eliciting and/or determining a cellular response, affecting biological processes, filtering fluids, as well as methods of screening, treatment and diagnosis. The biologically active surfaces can also be used in methods for preventing food contamination and/or spoilage. In some embodiments the immobilization of polysaccharides on substrates as provided herein is stable for at least 4 days. In still other embodiments the immobilization remains stable for at least 7 days.

In one aspect of the invention, therefore, a composition is provided, which comprises a polysaccharide, such as a glycosaminoglycan, immobilized on a substrate. In some embodiments, the immobilization occurs via hydrogen bonding. In one embodiment the polysaccharide is not hyaluronic acid. In another embodiment the polysaccharide is not heparin. In still another embodiment the polysaccharide is not hyaluronic acid or heparin.

In another aspect of the invention a composition is provided, which comprises a digested glycosaminoglycan immobilized on a substrate. In some embodiments the immobilization occurs via hydrogen bonding. In one embodiment the digested glycosaminoglycan is chemically digested, while in another embodiment the digested glycosaminoglycan is digested enzymatically with a glycosaminoglycan-digesting enzyme. The digested glycosaminoglycan in one embodiment is digested heparin or heparan sulfate.

In yet another aspect of the invention a composition is provided, which comprises at least two different polysaccharides (e.g., glycosaminoglycans) immobilized on a substrate. In one embodiment at least one glycosaminoglycan is immobilized to the substrate independently from another glycosaminoglycan (i.e., one glycosaminoglycan is not linked to the substrate via another glycosaminoglycan). The at least two different glycosaminoglycans can be immobilized on the substrate at different times, or they can be immobilized on the substrate at the same time. In one embodiment one of the at least two glycosaminoglycans is hyaluronic acid. In another embodiment one of the at least two glycosaminoglycans is a sulfated glycosaminoglycan. In some embodiments the sulfated glycosaminoglycan is a heparin/heparan sulfate-like glycosaminoglycan (HSGAG), a chondroitin sulfate glycosaminoglycan (CSGAG) or keratan sulfate. In other embodiments the sulfated glycosaminoglycan is a HSGAG, such as heparin or heparan sulfate.

In another aspect of the invention a composition is provided, which comprises one or more glycosaminoglycans immobilized on a substrate, wherein the substrate comprises polystyrene, an erethylene-benzene-containing-polymer or polyvinylidene chloride. In one embodiment the one or more glycosaminoglycans is a heparin/heparan sulfate-like glycosaminoglycan (HSGAG), a chondroitin sulfate glycosaminoglycan (CSGAG) or keratan sulfate. In another embodiment the one or more glycosaminoglycans comprise hyaluronic acid. In still another embodiment the one or more glycosaminoglycans comprise a digested glycosaminoglycan. In still another embodiment the one or more glycosaminoglycans comprise hyaluronic acid and a sulfated glycosaminoglycan. In yet another embodiment the one or more glycosaminoglycans are in an amount effective to prevent food contamination or spoilage.

The compositions provided herein, therefore, can be or form part of a food storage device. In one embodiment the food storage device is a wrap, such as a sheet or a film that can be used to cover or enclose food. In another embodiment the food storage device is a container into which food can be placed. Food storage devices, therefore, are also provided which comprise one or more immobilized glycosaminoglycans. The food storage devices can comprise glass, plastic, foam (e.g., Styrofoam®) or metal onto which one or more glycosaminoglycans are immobilized.

The compositions provided herein can also be or form part of a medical device. Therefore, in yet another aspect of the invention a medical device is provided, which comprises a glycosaminoglycan immobilized on a substrate, preferably, in some embodiments, via hydrogen bonding. In one embodiment the glycosaminoglycan is not hyaluronic acid. In another embodiment the glycosaminoglycan is not heparin.

In another aspect of the invention a medical device is provided, which comprises a digested glycosaminoglycan immobilized on a substrate. In one embodiment the digested glycosaminoglycan is immobilized via hydrogen bonding.

In still another aspect of the invention a medical device, which comprises at least two different glycosaminoglycans immobilized on a substrate, is provided. In one embodiment one of the at least two glycosaminoglycans is hyaluronic acid. In another embodiment one of the at least two glycosaminoglycans is a sulfated glycosaminoglycan.

The medical devices provided in one embodiment are implantable. In another embodiment the medical device is an extracorporeal medical device. In some embodiments the medical device is a tissue scaffold, stent, shunt, valve, pacemaker, pulse generator, cardiac defibrillator, spinal stimulator, brain stimulator, sacral nerve stimulator, lead, inducer, sensor, screw, anchor, pin, adhesion sheet, needle, lens, joint, prosthetic/orthopedic implant, catheter, tube (e.g., tubes for lines and drains) or suture.

The compositions provided herein can also be or form part of a filtering device. In one aspect of the invention, therefore, filtering devices are provided which can be used to filter fluids, such as, for example, body fluids (e.g., blood, cerebral spinal fluid (CSF), urine, etc.) In one embodiment the filtering devices are used to select a subset of cells. In yet another embodiment the filtering device removes metastatic cells from a body fluid. In still another embodiment the filtering devices are used to remove biological agents, such as proteins, glycoproteins, cells, infectious agents, etc. from the fluid. In one embodiment, therefore, the filtering devices are used to remove bacteria and/or viruses. In another embodiment the filtering device comprises chondroitin sulfate C.

The substrates can be hydrophobic or hydrophilic. In one embodiment the substrate is a hydrophobic substrate that has been modified to contain one or more hydrophilic groups. In another embodiment the hydrophilic groups comprise a silanol, carboxylic acid, hydroxyl group or some combination thereof.

In one embodiment the substrate is silicon oxide, glass, plastic, foam or metal. In another embodiment the substrate is a metal, such as, for example, steel (e.g., surgical or medical grade steel), titanium, palladium, chromium, calcium, zinc, iron, copper, gold or silver. In yet a further embodiment the substrate is a plastic, such as, for example, acrylonitrile butadiene styrene, polyamide 6,6 (Nylon), polyamide, polybutadiene, polybutylene terephthalate, polycarbonates, poly(ether sulphone) (PES, PES/PEES), poly(ether ether ketone)s, polyethylene (or polyethene), polyethylene glycol, polyethylene oxide, polyethylene terephthalate (PET, PETE, PETP), polyimide, polypropylene, polytetrafluoroethylene (Teflon) perfluoroalkoxy polymer resin (PFA), polystyrene, styrene acrylonitrile, poly(trimethylene terephthalate) (PTT), polyurethane (PU), polyvinylchloride (PVC), polyvinyldifluorine (PVDF), poly(vinyl pyrrolidone) (PVP), Kynar, Mylar, Rilsan, (e.g. polyamide 11 & 12), Ultem, Vectran, Viton and Zylon.

In another embodiment the substrate comprises polystyrene, an erethylene-benzene-containing polymer or polyvinylidene chloride. The polystyrenes can be injected, extruded, blow-molded or foamed. The substrates can, therefore, be wraps or foams.

The polysaccharides that are immobilized on the substrates can be any polysaccharide. In one embodiment the polysaccharide is a glycosaminoglycan. In another embodiment the glycosaminoglycan is a sulfated glycosaminoglycan. In still another embodiment the glycosaminoglycan is sulfated hyaluronic acid. The glycosaminoglycan, in yet another embodiment, is a heparin/heparan sulfate-like glycosaminoglycan (HSGAG), a chondroitin sulfate glycosaminoglycan (CSGAG) or keratan sulfate. In still another embodiment the glycosaminoglycan is a HSGAG, such as heparin or heparan sulfate. In yet another embodiment the glycosaminoglycan is a CSGAG, such as chondroitin sulfate or dermatan sulfate. In still a further embodiment the chondroitin sulfate or dermatan sulfate is chondroitin sulfate A, chondroitin sulfate B or chondroitin sulfate C. In another embodiment the glycosaminoglycan is not hyaluronic acid. In still another embodiment the glycosaminoglycan is not heparin.

The polysaccharides for use in the compositions, devices and methods provided can also be digested polysaccharides. In one embodiment the digested polysaccharide is digested via chemical digestion. In another embodiment the digested polysaccharide is digested via enzymatic digestion. In one embodiment the digested polysaccharide is a digested glycosaminoglycan. In another embodiment the digested glycosaminoglycan is a digested HSGAG, CSGAG or keratan sulfate. In still another embodiment the digested glycosaminoglycan is digested heparin, heparan sulfate, chondroitin sulfate or dermatan sulfate. In still another embodiment the digested glycosaminoglycan is digested chondroitin sulfate A, chondroitin sulfate B or chondroitin sulfate C. In one embodiment a polysaccharide is immobilized on a substrate in non-digested form but is digested after immobilization.

As provided above, the digested polysaccharide can be produced via enzymatic digestion. Therefore, the digested glycosaminoglycans can be produced with the use of a glycosaminoglycan-degrading enzyme. In one embodiment the glycosaminoglycan-degrading enzyme is a heparinase, chondroitinase, sulfatase, sulfotransferase, glycuronidase, iduronidase, glucuronidase or keratanase. In another embodiment the glycosaminoglycan-degrading enzyme is a heparinase, such as heparinase I, heparinase II or heparinase III. In still another embodiment the glycosaminoglycan-degrading enzyme is a chondroitinase, such as chondroitinase AC, chondroitinase ABC (e.g., chondroitinase ABC I, chondroitinase ABC II) or chondroitinase B.

The compositions provided can include one or more kinds of glycosaminoglycans in some embodiments. In one embodiment, therefore, compositions are provided wherein an additional glycosaminoglycan is immobilized on the substrate. In other embodiments the compositions provided can include one or more additional biological agents, such as proteins, glycoproteins, cells, lipids, etc. In some embodiments the protein or glycoprotein is fibronectin, hydroxyappetite, a collagen, an integrin, an adhesin, a proteoglycan, a growth factor or a cytokine. In still other embodiments the compositions provided further comprise at least one therapeutic agent. In one embodiment the therapeutic agent is a biological agent. In still another embodiment the therapeutic agent is a drug. In some embodiments additional polysaccharides (e.g., glycosaminoglycans), biological agents or therapeutic agents are immobilized on the substrate via hydrogen bonding. In other embodiments additional polysaccharides (e.g., glycosaminoglycans), biological agents or therapeutic agents are immobilized via covalent attachment to the substrate. In one embodiment covalent attachment can be achieved via a linking molecule. In still other embodiments additional polysaccharides (e.g., glycosaminoglycans), biological agents or therapeutic agents are immobilized via binding to a ligand, such as an antibody. In still further embodiments additional polysaccharides (e.g., glycosaminoglycans), biological agents or therapeutic agents are immobilized via binding to the immobilized polysaccharides.

The compositions and devices provided can be used in some aspects of the invention for a variety of purposes and in a variety of methods. In one aspect the compositions and devices promote the adhesion of proteins or cells. In another aspect the compositions and devices resist the adhesion of proteins or cells. In still another aspect the compositions and devices promote the proliferation of cells. In still a further aspect the compositions and devices inhibit the proliferation of cells. In yet another aspect the compositions and devices inhibit bacterial or viral adhesion. In still another aspect the compositions and devices promote bacterial or viral adhesion.

In one embodiment, therefore, the compositions and devices provided can include a glycosaminoglycan with any of the above-mentioned properties. In one embodiment the glycosaminoglycan that inhibits protein binding is hyaluronic acid, heparin, heparan sulfate, chondroitin sulfate A, chondroitin sulfate C, dermatan sulfate, heparinase I-digested heparin, heparinase I-digested heparan sulfate, heparinase III-digested heparin, heparinase III-digested heparan sulfate or some combination thereof. In another embodiment the glycosaminoglycan that resists cell adhesion is hyaluronic acid, dermatan sulfate, heparinase III-digested heparin or some combination thereof. In still another embodiment the glycosaminoglycan that promotes cell adhesion is heparin, heparan sulfate, chondroitin sulfate C, chondroitin sulfate A, dermatan sulfate, heparinase I-digested heparin, heparinase I-digested heparan sulfate, heparinase III-digested heparan sulfate or some combination thereof. In yet another embodiment the glycosaminoglycan that promotes proliferation is chondroitin sulfate C, dermatan sulfate, heparan sulfate, heparinase I-digested heparin, heparinase III-digested heparin or some combination thereof. In a further embodiment the glycosaminoglycan that inhibits proliferation is hyaluronic acid, chondroitin sulfate A, heparin, heparinase I-digested heparan sulfate, heparinase III-digested heparan sulfate or some combination thereof. In yet a further embodiment the glycosaminoglycan inhibits cancer cell growth. Such glycosaminoglycans include chondroitin sulfate C, dermatan sulfate, heparan sulfate, heparinase I-digested heparan sulfate, heparinase III-digested heparin, heparinase III-digested heparan sulfate or some combination thereof. In another embodiment the glycosaminoglycan that inhibits cell migration or metastasis is dermatan sulfate, heparinase III-digested heparan sulfate, hyaluronic acid, chondroitin sulfate C, heparinase I-digested heparin, heparinase I-digested heparan sulfate, heparinase III-digested heparin or some combination thereof. In still another embodiment the glycosaminoglycans that can promote bacterial or viral adhesion are HSGAGs, such as heparin or heparan sulfate.

Surfaces can be created with more than one biological property. For instance, in one embodiment, a surface can be created that promotes cell adhesion and cell growth (proliferation). In another embodiment a surface can be created that promotes cell adhesion and inhibits cell growth. These surfaces can be created by immobilizing glycosaminoglycans that exhibit multiple properties. For instance, glycosaminoglycans that promote cell adhesion and cell growth include chondroitin sulfate C, dermatan sulfate, heparan sulfate, heparinase I-digested heparin and heparinase III-digested heparin. Glycosaminoglycans that promote cell adhesion and inhibit metastasis or proliferation include heparinase III-digested heparan sulfate, heparin, heparinase I-digested heparan sulfate, hyaluronic acid and chondroitin sulfate A. These glycosaminoglycans can be used in some embodiments to treat cancer. In another embodiment surfaces with more than one biological property can be created by immobilizing a combination (i.e., more than one) of different glycosaminoglycans.

Biologically active surfaces can be created on not only food storage and medical devices, such as implantable medical devices, but also on particles (e.g., inhalable particles, particles for oral or rectal delivery, etc.), pills as well as on slow release drug delivery vehicles.

The compositions and devices provided can be used in a variety of methods of treatment. In one aspect of the invention compositions and methods for treating cancer are provided. In one embodiment the composition comprises an amount of a glycosaminoglycan effective for treating cancer. In another embodiment the glycosaminoglycan is a HSGAG. In still another embodiment the glycosaminoglycan is a heparinase III-digested HSGAG. In another embodiment the cancer is skin or ovarian cancer.

In another aspect of the invention compositions and methods for inhibiting or promoting angiogenesis are provided. In still another aspect of the invention compositions and methods for treating a neurodegenerative disorder are provided. In one embodiment the neurodegenerative disorder is a neurodegenerative disease. In another embodiment the neurodegenerative disorder is a central nervous system injury. In yet another embodiment the central nervous system injury is a spinal cord injury. In yet another aspect of the invention compositions and methods for preventing infection are provided. In still another aspect of the invention compositions and methods for promoting implant adhesion are provided. In still a further aspect of the invention compositions and methods for preventing infection or preventing the attachment of infectious agents to a medical device are provided. In yet another aspect of the invention compositions and methods for wound healing are provided. In still another aspect of the invention compositions and methods for preventing inflammation are provided. In another aspect of the invention compositions and methods for inhibiting coagulation or treating a disease associated with coagulation are provided. In still another aspect of the invention compositions and methods for the treatment of cystic fibrosis are provided.

Glycosaninoglycans, such as HSGAGs, are useful for the therapeutic endpoints provided herein. Therefore, in one embodiment the compositions provided contain an effective amount of a glycosaminoglycan for the particular therapeutic endpoint desired. In another embodiment the compositions provided further comprise an agent in addition to the immobilized glycosaminoglycan, such as a therapeutic agent, and it is the therapeutic agent that is in an effective amount for reaching the desired therapeutic endpoint. In still another embodiment the composition comprises an additional therapeutic agent, and it is the combination of the glycosaminoglycan and the additional agent that is effective. The compositions and devices provided can be used to treat any of the diseases or disorders described herein. Methods of using the compositions and devices for treating a subject with any of the diseases or disorders described herein are also provided.

In one aspect of the invention a method of treating a subject with cancer by administering a composition or device as described above is provided. In another aspect of the invention a method of treating a subject with a neurodegenerative disorder is provided. In still another aspect of the invention a method of treating a subject with an infection is provided. In a further aspect of the invention a method of treating a subject with an infection is provided. In one embodiment the device administered to the subject is a medical device as provided herein.

In another aspect of the invention a method is provided whereby a subject is treated by administering a medical device with or without glycosaminoglycans immobilized thereon and administering one or more glycosaminoglycans as a separate step. In one embodiment the glycosaminoglycans can be administered subsequent to or concomitantly with the administration of the medical device. In another embodiment the medical device is implanted in the subject. In still another embodiment the glycosaminoglycan is administered to the subject's blood stream. In one embodiment the administration of the glycosaminoglycan is intravenous administration.

The compositions provided herein can also be used to prevent food contamination or spoilage. In one aspect of the invention a food is contacted with any of the compositions or devices provided herein in order to prevent food contamination or spoilage. In one embodiment the food is a meat or produce. In another embodiment the meat is beef, poultry or fish. In still another embodiment the produce is a vegetable or fruit. In one embodiment the contacting can be carried out by placing the food inside a food storage device. In another embodiment the food is covered or wrapped with a food storage device.

The compositions provided can also be used in a variety of screening and/or diagnostic methods. In one aspect of the invention a method of screening a cell or subcellular preparation by contacting a composition as provided herein with a cell or subcellular preparation and testing the cell or subcellular preparation to identify a response is provided. In one embodiment the response is binding of the cell or subcellular preparation or a component thereof to at least one glycosaminoglycan of the composition. In another embodiment the response is the proliferation of cells. In still another embodiment the response is the migration of cells. In yet another embodiment the response is adhesion of a cell or a component of the subcellular preparation to at least one glycosaminoglycan of the composition. In one embodiment the cell or subcellular preparation is contacted with an agent, such as a therapeutic agent, prior to contact with the composition. In another embodiment the cell preparation is two or more cell populations. In yet another embodiment the two or more cell populations are dissimilar cell populations. In still another embodiment the testing of the response allows for the comparison or separation of two cell populations.

Also provided in another aspect of the invention are methods of determining a cellular response by contacting a composition provided herein with a cell preparation and measuring a marker for a cellular response. In one embodiment the amount of a nucleic acid or protein or the phosphorylation state of a protein is measured. In another embodiment the marker is a marker for proliferation or adhesion. In one embodiment the marker is a proliferative protein (e.g., ERK, MEK, etc.), an adhesion-related protein (e.g.,CD44, FAK, etc.) or an apoptosis-related protein (e.g., Akt/PKB, caspases, etc.).

In another aspect of the invention methods for producing substrates with immobilized polysaccharides thereon are also provided. In one embodiment the method includes the introduction of hydrophilic groups to a substrate. In another embodiment the method includes the introduction of charged nitrogens, oxygens, etc. to the surface of a substrate. In one embodiment the introduction of charged nitrogens, oxygens, etc. is accomplished by plasma cleaning. In another embodiment it is accomplished by changing the pH. In still another embodiment the method further includes contacting the substrate with a polysaccharide, such as a HSGAG.

In another aspect of the invention a method of immobilizing polysaccharides (e.g., glycosaminoglycans) on a substrate is provided. In one aspect a glycosaminoglycan is immobilized by contacting a substrate with the glycosaminoglycan. In one embodiment the substrate is positively charged or neutral. In another embodiment the substrate is contacted with the glycosaminoglycan in acidic or neutral conditions (i.e., acidic or neutral pH). In still another embodiment the substrate is contacted with the glycosaminoglycan for at least 30 minutes prior to washing. In still other embodiments the substrate is contacted with the glycosaminoglycan for 1, 2, 3, 4, 5, 7, 10, 12, 15, 20, 24 or more hours prior to washing. In yet another embodiment the substrate is contacted with the glycosaminoglycan and allowed to dry prior to washing. In still a further embodiment the substrate is cleaned prior to contact with the glycosaminoglycan. In another embodiment the substrate is cleaned (e.g., with O₂ plasma) prior to contact with the glycosaminoglycan. In still another embodiment hydrophilic groups are created on the surface of the substrate prior to contact with the glycosaminoglycan. In yet another embodiment —OH groups are created on the surface of the substrate (e.g., a glass substrate) prior to contact with the glycosaminoglycan.

The immobilization of the polysaccharides on the substrates, in some embodiments, is stable for 1, 2, 3, 4, 7, 10, 14, 20 or more days.

Each of the limitations of the invention can encompass various embodiments of the invention. It is, therefore, anticipated that each of the limitations of the invention involving any one element or combinations of elements can be included in each aspect of the invention. These and other aspects of the invention, as well as various advantages and utilities, will be more apparent with reference to the detailed description of the preferred embodiments.

### BRIEF DESCRIPTION OF THE FIGURES

FIG. 1 provides the high-resolution XPS spectra for (a) nitrogen (N Is) and (b) carbon (C 1s) peaks in HA recorded for as-spun, washed and bare silicon oxide substrates. For carbon peaks of an as-spun and a washed film, the spectra were deconvoluted with four Gaussian peaks that are assigned at each oxidized state. For convenience, the peak for the strongly oxidized carbon (CO*) was not deconvoluted in detail. All films were prepared and characterized on the silicon oxide substrate to take advantage of the flat surface.

FIG. 2 shows the wide scans of XPS spectra for as-spun, washed and bare silicon oxide substrates. The results indicate that the substrate surface is nearly fully covered with the chemisorbed layer.

FIG. 3 provides the AFM images of surface roughness and the corresponding fluorescent images for FN adsorption for (a) a bare silicon oxide substrate, (b) a HA surface after thorough washing and (c) an as-coated HA film. The roughness of the film after washing is less than unwashed films but greater than substrate alone, supporting the presence of a chemisorbed layer. The height scale is 5 nm and the scan size is 1×1 μm². The fluorescent images reveal that the surface is fully covered with HA even after extensive washing.

FIG. 4 provides the amount of FN adsorption onto GAG surfaces, which was measured by quantifying the fluorescence intensity. The results are normalized to glass (defined as 100) as the positive control and no protein (defined as 0). Data are presented as a percentage of the difference between untreated and glass. * denotes p<0.05 compared to glass, and +denotes p<0.05 compared to HA.

FIG. 5 illustrates the stability of the HA surface examined by the quantitative analysis of protein adsorption as a function of exposure times to PBS prior to exposure and subsequent staining to FN. Note that the surface was stable and greatly reduced protein adsorption by more than 92% even after exposure to PBS for up to 7 days. No contrast enhancement was made throughout the analysis. * denotes p<0.05.

FIG. 6 shows that GAG families can be deposited to create surfaces. FIG. 6A provides the structures of disaccharides composing the various GAGs used. The HSGAG disaccharide can be modified at five sites. Three sites (2-0, 3-0, and 6-0) indicated by “X” can be sulfated. The site denoted by “Y” can be unmodified, acetylated or sulfated. The epimerization state of C5 sugar of the uronic acid determines whether iduronic acid or glucuronic acid is present. Heparin is a highly sulfated HSGAG while HS is an undersulfated HSGAG. The chondroitin sulfate disaccharide is specifically sulfated to determine its species. CS A is sulfated at X_{A} and unmodified at X_{C}, while CS C is sulfated at X_{C} and unmodified at X_{A}. The dermatan disaccharide can be sulfated at additional sites to that illustrated. The epimerization state of C5 sugar of the uronic acid determines whether iduronic acid or glucuronic acid is present. FIG. 6B provides the contact angles measured for water on various GAG surfaces. Left and right contact angles were averaged, and data are presented in degrees. Untreated refers to silicon dioxide without GAG. * denotes p<0.05 for a GAG surface compared to untreated of the same washing state. † denotes p<0.05 for a GAG surface compared to HA of the same washing state. § denotes p<0.05 for the washed surface compared to the unwashed surface for a given GAG.

FIG. 7 demonstrates that GAGs can be immobilized to create surfaces. Contact angles for water on various GAG surfaces were measured. Left and right contact angles were averaged, and data are presented in degrees. * denotes p<0.05 for a GAG surface compared to silicon dioxide (untreated).

FIG. 8 illustrates that GAG surfaces resist protein binding. FN adsorption onto GAG surfaces was measured by quantifying the fluorescence intensity. The resistance of FN binding was determined by normalizing the intensity results to glass (defined as 0) and no protein (defined as 100). Data are presented as the percent reduction in bound FN from glass, which readily binds FN. * denotes p<0.05 compared to glass, and † denotes p<0.05 compared to HA.

FIG. 9 illustrates that GAG surfaces inhibit protein adhesion. FN adsorption onto GAG surfaces was measured by quantifying the fluorescence intensity. The resistance of FN binding was determined by normalizing the intensity results to glass (defined as 0) and no FN treatment (defined as 100). Data are presented as the percent reduction in bound FN compared to glass, which readily binds FN. * denotes p<0.05 compared to glass, and † denotes p<0.05 compared to HA.

FIG. 10 shows that GAG surfaces regulate cell adhesion and proliferation. FIG. 10A provides the results from the GAG surfaces that were created on glass. B16F10 cells were added to surfaces, and whole cell number was determined at 2, 24, 48, 72, and 96 hours. FIG. 10B provides the results from adding B16F10 cells to GAG surfaces formed on glass. The percentage of cells adhered after 2 hours was determined by measuring whole cell count. * denotes p<0.05 for various surfaces compared to glass. FIG. 10C provides results from the addition of B16F10 cells to GAG surfaces. Whole cell numbers were determined after 1 and 4 days. Bars represent the percentage of the cells on day 1 that were present on day 4. The numbers are the average growth rate per day. * denotes p<0.05 for various surfaces compared to glass.

FIG. 11 demonstrates that GAG surfaces modulate cell adhesion. B16-FI0 cells were added to GAG surfaces formed on glass. The percentage of cells adhered after 2 hours was determined by measuring whole cell count. * denotes p<0.05 compared to glass. † denotes p<0.05 compared to FN.

FIG. 12 illustrates that GAG surfaces regulate proliferation. GAG surfaces were created on glass, B16-F10 cells were added to surfaces and whole cell number was determined at 2, 24, 48, 72, and 96 hours by measuring whole cell count. Data are presented as percent change in whole cell number after 96 hours compared to the number of cells adhered. Numbers illustrate the percent growth per day. * denotes p<0.05 compared to glass. † denotes p<0.05 compared to FN.

FIG. 13 shows that GAG surfaces alter FAK and CD44 expression. B16F 10 cells were deposited on GAG surfaces. Cells were fixed after 24 hours. Immunohistochemistry was performed for FAK (green) and CD44 (red) using appropriate antibodies, as well as for cell nuclei (blue) using DAPI. The “combined” row represents an overlay of immunohistochemical results for all three markers.

FIG. 14 illustrates that GAG surfaces alter FAK and CD44 expression. B16-F10 cells were immobilized on GAG surfaces. Cells were fixed after 24 hours. Immunohistochemistry was performed for FAK (green) and CD44 (red) using appropriate antibodies, as well as for cell nuclei (blue) using DAPI. The “combined” row represents an overlay of immunohistochemical results for all three markers.

FIG. 15 shows that GAG surfaces influence cellular proliferation distinct from free GAGs. B16F10 cells were treated with PBS or GAGs at various concentrations. Whole cell number was determined after 72 hours, and data were normalized by the percent of cells remaining in GAG treated conditions compared to the PBS treated condition. FIG. 15A provides the characterization of the effects of distinct GAG types. FIG. 15B illustrates the effects of whole and digested HSGAGs.

FIG. 16 shows that immobilized GAGs regulate proliferation distinct from free GAGs. B 16-F10 cells were treated with GAGs, and whole cell number was determined after 72 hours. Data were normalized as the percent of cells in GAG treated conditions compared to the PBS treated condition. Results are presented as non-HSGAG-treated (left) and HSGAG-treated (right) conditions to aid in visualization of the results.

FIG. 17 illustrates that heparinase digested HSGAGs can be deposited to create surfaces that regulate biological processes. FIG. 17A provides the results from the measurement of the contact angles for water on HA surfaces and on various HSGAG surfaces. Left and right contact angles were averaged and data are presented in degrees. Untreated refers to silicon dioxide without GAG. * denotes p<0.05 for a GAG surface compared to untreated of the same washing state. † denotes p<0.05 for a GAG surface compared to HA of the same washing state. § denotes p<0.05 for the washed surface compared to the unwashed surface for a given GAG. FIG. 17B provides the results from the measurement of FN adsorption onto HA and HSGAG surfaces by quantifying the fluorescence intensity. The resistance of FN binding was determined by normalizing the intensity results to glass (defined as 0) and no protein (defined as 100). Data are presented as the percent reduction in bound FN from glass, which readily binds FN. * denotes p<0.05 compared to glass, and † denotes p<0.05 compared to HA. FIG. 17C provides results from the HSGAG surfaces created on glass. B16F10 cells were added to surfaces, and whole cell number was determined at 2, 24, 48, 72, and 96 hours. FIG. 17D provides the results from the addition of B 16F10 cells to HSGAG surfaces formed on glass. The percentage of cells adhered after 2 hours was determined by measuring whole cell count. * denotes p<0.05 for digested heparin compared to undigested heparin. † denotes p<0.05 for digested HS compared to undigested HS surfaces compared to glass. FIG. 17E provides the results from the addition of B16F10 cells to HSGAG surfaces. Whole cell numbers were determined after 1 and 4 days. Bars represent the percentage of the cells on day 1 that were present on day 4. The numbers are the average growth rate per day. * denotes p<0.05 for digested heparin compared to undigested heparin. † denotes p<0.05 for digested HS compared to undigested HS.

FIG. 18 illustrates that surfaces formed by digested HSGAGs alter FAK and CD44 expression. B16F10 cells were deposited on HSGAG surfaces. Cells were fixed after 24 hours. Immunohistochemistry was performed for FAK (green) and CD44 (red) using appropriate antibodies, as well as for cell nuclei (blue) using DAPI. The “combined” row represents an overlay of immunohistochemical results for all three markers.

FIG. 19 demonstrates the stability of the adsorbed HA on a glass substrate measured by the quantitative analysis of the adsorption of FITC-labeled BSA. The results were normalized relative to glass controls. HA was stable for at least 7 days in all conditions. HA dissolved either in PBS (▪) or water (□) produced surfaces that remained stable for at least 14 days when exposed to air. More than 60% of the HA dissolved in PBS (●) and water (◯) detached after 10 days and 14 days of exposure to PBS. The values indicate the mean of four independent experiments. Error bars indicate SD.

FIG. 20 demonstrates that a thin film of HA was spin-coated on to medical grade steel plates. The HA was allowed to settle and dry. HA attachment was measured by determining the ability of the coated surface to resist fluorescent BSA binding. HA-coated steel (FIG. 20A); steel alone (FIG. 20B) and unstained steel (FIG. 20C).

### DETAILED DESCRIPTION OF THE INVENTION

Polysaccharides have a number of potential applications, such as, for example, in biomedical applications in drug delivery and tissue engineering. For these applications, it is important to understand the characteristics of polysaccharide films directly immobilized to solid substrates. It has now been discovered that a variety of glycosaminoglycans, in addition to hyaluronic acid, can be efficiently immobilized on substrates, such as, for example, hydrophilic substrates, and that such surfaces can influence biological activity.

The invention, therefore, in one aspect provides substrates with immobilized polysaccharides thereon. The polysaccharides for use in the compositions provided herein can be any molecule which contains two or more consecutively linked monosaccharides. Polysaccharides include those that are isolated from plant, animal and microbial sources as well as those that are synthetic. The term “polysaccharide” as used herein, therefore, includes mucins, alginates, pectins, fucoidans, carrageenans, chitin, pentosan, dextran sulfate, laminarin, fucans, glucans, calcium spirulan, xylan, amylose, cellulose, curdlan, trehalose, glycans, mannitol, galactose, sucrose and D-galactan. Preferably, the polysaccharides are glycosaminoglycans (GAGs). Glycosaminoglycans are a family of complex polysaccharides that include, for example, dermatan sulfate (DS), chondroitin sulfate (CS), heparin, heparan sulfate (HS), keratan sulfate and hyaluronic acid (HA). The term “polysaccharide”, therefore, also refers to sulfated or highly sulfated glycosaminoglycans. In one embodiment, therefore, the polysaccharide is sulfated, such as a sulfated glycosaminoglycan, and is not, therefore, hyaluronic acid. The glycosaminoglycans can have a high molecular weight and/or high charge density. Other examples of glycosaminoglycans include sulfated hyaluronic acid, heparin/heparan sulfate-like glycosaminoglycans (HLGAGs/HSGAGs), biotechnologically prepared heparin, chemically modified heparin, synthetic heparin, heparinoids, enoxaparin, low molecular weight heparin (LMWH) or specific kinds of chondroitin sulfate, such as chondroitin sulfate A (CS A), chondroitin sulfate B (CS B) or chondroitin sulfate C (CS C). Polysaccharides, in some embodiments, may also include heparin-like polyanions which are similar to heparin and are naturally occurring or synthetic. Such heparin-like polyanions include poly(vinyl sulfate) and poly(anethole sulfonate).

Polysaccharides can also be modified versions of the polysaccharides provided herein. These “modified polysaccharides” can be modified by depolymerization, phosphorylation, sulfonation, regioselective sulfonation and/or desulfonation. In particular, modified polysaccharides include polysaccharides that have been modified with chemical degradation (e.g., periodate oxidation and base cleavage, alkaline degradation, nitrous acid cleavage) or enzymatic degradation (i.e., with polysaccharide-degrading enzymes).

“Polysaccharide degrading enzymes” are enzymes that cleave, degrade or somehow modify a polysaccharide when placed in contact with the polysaccharide. Polysaccharide degrading enzymes include but are not limited to, chondroitinases (e.g. chondroitinase AC (cAC), chondroitinase B (cB), chondroitinase ABC (cABC)), hyaluronate lyase, heparinases (e.g., heparinase I (hepI), heparinase II (hepII), heparinase III (hepIII)), keratanase, D-glucuronidase, L-iduronidase, glycuronidases (e.g., Δ4,5 glycuronidase), sulfatases (e.g., 2-O sulfatase, 3-O sulfatase, 6-O sulfatase), C5-epimerase, sulfotransferases, (e.g., 2-O sulfotransferase, 3-O sulfotransferase, 6-O sulfotransferase, N-sulfotransferase (NDST)), modified versions of these enzymes, variants and functionally active fragments thereof. Polysaccharide-degrading enzymes, therefore, include glycosaminoglycan-degrading enzymes; and, therefore, in one aspect of the invention substrates are provided which include immobilized polysaccharides that are digested glycosaminoglycans. The immobilized polysaccharides in this aspect of the invention can be digested prior to or after their immobilization.

In addition, in some embodiments the modified polysaccharides are sulfated versions of a polysaccharide provided herein. Examples of such sulfated polysaccharides include sulfated D-galactan, sulfated α-(1-3)-D-glucan, laminarin sulfate, natural sulfated fucans, sulfated hyaluronic acid, etc.

As used herein a “substrate” can be any substrate on which one or more polysaccharides can be immobilized. The substrate can be hydrophobic or hydrophilic. A “hydrophilic substrate” is intended to include materials that are naturally, without modification, hydrophilic in nature (i.e., have hydrophilic functional groups) as well as materials that are not naturally hydrophilic but are modified to be so. One of ordinary skill in the art is familiar with methods that can be used to modify otherwise non-hydrophilic substrates. For instance, it will be readily appreciated that non-hydrophilic substrates, such as hydrophobic polystyrene, can be chemically modified to include hydrophilic groups like silanol (—SiOH), carboxylic acid or hydroxyl groups. This chemical modification could occur either from chemical reactions occurring at the surface as a result of solvent or vapor reactions, such as through surface treatment with oxygen plasma.

Examples of substrates that can be used in the compositions, devices and methods provided herein include, for example, include glass, silicon oxides, plastics, foams or metals. Plastic substrates include, for example, acrylonitrile butadiene styrene, polyamide (Nylon), polyamide, polybutadiene, polybutylene terephthalate, polycarbonates, poly(ether sulphone) (PES, PES/PEES), poly(ether ether ketone)s, polyethylene (or polyethene), polyethylene glycol, polyethylene oxide, polyethylene terephthalate (PET, PETE, PETP), polyimide, polypropylene, polytetrafluoroethylene (Teflon) perfluoroalkoxy polymer resin (PFA), polystyrene, styrene acrylonitrile, poly(trimethylene terephthalate) (PTT), polyurethane (PU), polyvinylchloride (PVC), polyvinyldifluorine (PVDF), poly(vinyl pyrrolidone) (PVP), Kynar, Mylar, Rilsan, (e.g. polyamide 11 & 12), Ultem, Vectran, Viton and Zylon. Substrates further include but are not limited to membranes, e.g., natural and modified celluloses such as nitrocellulose or nylon, sepharose, agarose, polystyrene, polypropylene, polyethylene, dextran, amylases, polyacrylamides, polyvinylidene difluoride, PEGylated or calcium alginate spheres, other agaroses and magnetite, including magnetic beads. Substrates also include coblock polymers, which have both hydrophilic and hydrophobic components. Substrates further include those that comprise erethylene-benzene containing polymers and polyvinylidene chloride. As used herein, “erethylene-benzene containing polymers” are any polymer that contain erethylene and benzene in some number and combination. Therefore, included in this group are polymers that form foams, such as Styrofoamg. Accordingly, the substrates provided herein also include foam, such as Styrofoamg. Polyvinylidene chlorides include polymerized vinylide chlorate containing monomers of acrylic esters and unsaturated carboxyl groups. The substrates provided herein, therefore, also include wraps, such as sheets or films, that contain polyvinylidene chloride.

As provided above, in some embodiments the substrate is hydrophilic. Hydrophilic substrates include, for example, glass, silicon oxides, some plastics and some metals. Hydrophilic metal substrates include steel, palladium, chromium, calcium, zinc, copper, iron, gold or silver. The metals provided herein further include medical grade or surgical steel.

The substrate can be totally insoluble or partially soluble and may have any possible structural configuration. Thus, the substrate may be conical, hemispherical, as in an orthopedic implant, spherical, as in a bead, string-like (braided or unbraided), as in sutures, or cylindrical, as in the inside or outside of tubing, the surface of a test tube or microplate well, or the external surface of a rod. Alternatively, the surface may be flat such as a sheet, film, test strip, bottom surface of a microplate well, drain, etc. The substrates can also be part of or in the form of a container. “Containers” as used herein refer to any container of any shape that can hold another substance, such as a food. Containers, therefore, include cups, bowls, bags, cans, thermoses, or any other food storage device.

It has been demonstrated that polysaccharides, such as heparin, heparan sulfate, chondroitin sulfate, dermatan sulfate and high molecular weight HA, can be directly immobilized onto substrates, such as hydrophilic substrates. It has also been demonstrated that although polysaccharides can be immobilized using any method known to those in the art, which include covalent bonding, crosslinking, linkage via a ligand, etc., polysaccharides can also be immobilized without any chemical manipulation, allowing for the formation of an ultra-thin chemisorbed layer. The polysaccharides that are immobilized with such a method are stabilized on hydrophilic surfaces through hydrogen bonding between the hydrophilic moieties of the polysaccharides (such as carboxylic acid (—COOH) or hydroxyl (—OH) groups) with silanol (—SiOH), carboxylic acid or hydroxyl groups on the substrates. Therefore, substrates are provided, in one embodiment, whereby the polysaccharides are immobilized via hydrogen bonding. Preferably, the hydrogen bonding is predominant in immobilizing the polysaccharides to the substrate, or in other words, the majority of polysaccharide immobilization is accomplished via hydrogen bonding. In some embodiments at least 50%, 55%, 60%, 65%, 70%, 75%, 80%, 85%, 90%, 95%, 99%, or more of the polysaccharides immobilized on the substrate are immobilized via hydrogen bonding.

Hydrogen bonding can be accomplished by making or introducing charged nitrogens and/or oxygens on a substrate, which can be done with a variety of techniques, which include, plasma cleaning, altering the pH, running an electric current through the substrate, putting the substrate in a magnetic field, introducing agents that would increase the number of charged groups (nitrogen/oxygen/sulftir, etc.) on the substrate or resynthesizing the substrate with a high concentration of these compounds, etc. A preferred method to accomplish hydrogen bonding immobilization is provided herein and given below in the Examples. In another embodiment, substrates are provided whereby the majority of the polysaccharide immobilization does not occur via hydrogen bonding. In some embodiments, therefore, at least about 50%, 55%, 60%, 65%, 70%, 75%, 80%, 85%, 90%, 95%, 99%, or more of the polysaccharide immobilization is accomplished via non-hydrogen bonding directly to the surface. Such bonding includes covalent bonding, crosslinking between polysaccharides, linkage via a ligand, etc. In these embodiments, the non-hydrogen bonding can be combined with hydrogen bonding, provided that the hydrogen bonding represents only about 5%, 10%, 15%, 20%, 25%, 30%, 35%, 40% or 45% of the polysaccharide immobilization.

Furthermore, it has been found that, despite the water solubility, chemisorbed polysaccharide layers (those created predominantly via hydrogen bonding) remained stable on, for example, hydrophilic glass or silicon oxide substrates. For instance, chemisorbed HA layers were stable for at least 7 days in phosphate buffered saline, while other glycosaminoglycans have been found to be stable for at least 4 days. Therefore, in one embodiment, compositions are provided, which include polysaccharides immobilized on a substrate, wherein the immobilized polysaccharide layers are stable for at least 1, 2, 3, 4, 5, 6, 7, 10, 14, 21, 30 or more days.

Biologically active surfaces (i.e., substrates with polysaccharides immobilized thereon and which have some biological activity), in another aspect of the invention, can also include other biological or therapeutic agents. Therefore, substrates are provided on which two or more different kinds of polysaccharides, such as two or more different kinds of glycosaminoglycans, are immobilized. Biologically active surfaces with 2, 3, 4, 5, 6, 7, 8, 9, 10 or more different kinds of polysaccharides (e.g., glycosaminoglycans) are, therefore, provided. In one embodiment at least one of the polysaccharides is immobilized on the substrate independently from another polysaccharide (i.e., the immobilization of at least one of the polysaccharides does not occur through linkage with another immobilized polysaccharide). In one embodiment, one of the at least two polysaccharides is hyaluronic acid. In another embodiment one of the at least two polysaccharides is a sulfated glycosaminoglycan. Therefore, substrates that contain both hyaluronic acid and a sulfated glycosaminoglycan are also provided.

“Biological agents”, as used herein, include, in addition to polysaccharides, nucleic acids, proteins, peptides, glycoproteins, lipids, cells, etc. The biological agent can also be a therapeutic agent. The biological agents can be bound to the substrate, for example, directly, via a linker (e.g., a bifunctional linker), or via binding to another biological agent immobilized on the substrate (e.g., a ligand, such as an antibody).

In another aspect of the invention the substrates provided include one or more polysaccharides and one or more non-polysaccharide biological agents. In a preferred embodiment the non-polysaccharide biological agent is a glycoprotein, protein or a biologically active fragment thereof. “Biologically active”, as used herein, refers to a function possessed by a polysaccharide or other agent. In some embodiments, when the term is used to characterize a fragment, it is meant to refer to a fragment that possesses some biological function. Proteins or glycoproteins for use in the substrates and methods of the invention include fibronectin, hydroxyappetite, collagens, integrins, adhesins, proteoglycans, growth factors, cytokines, etc. In another preferred embodiment the biological agent is a cell or a population of cells. Therefore, the substrates provided can firther include one or more cell populations of similar or dissimilar origin. The cells can be adhered to the substrates of the invention via any method known to those of ordinary skill in the art. In one embodiment the cell or cells can adhere to the substrate by binding to the biological agents present on the substrate. The cells can bind to the polysaccharides, the non-polysaccharide biological agents or both. Substrates of the invention, therefore, also can include ligands to which the cells or component of the cells (e.g., a surface receptor) bind. Because of the ability to choose which polysaccharides and/or other biologic agents to immobilize on a substrate as well as the pattern of immobilization, the location of desired biological properties, such as the location of protein or cell adhesion, is controllable.

In some embodiments the biological agents provided herein are in a substantially pure form. As used herein, with respect to these molecules, the term “substantially pure” means that the molecules of the invention are essentially free of other substances with which they may be found in nature or in vivo systems to an extent practical and appropriate for their intended use. In particular, the molecule is sufficiently free from other biological constituents of their hosts cells so as to be useful in, for example, producing pharmaceutical preparations. Because the molecules of the invention may be admixed with a pharmaceutically acceptable carrier in a pharmaceutical preparation, the molecule may comprise only a small percentage by weight of the preparation. The molecule is nonetheless substantially pure in that it has been substantially separated from the substances with which it may be associated in living systems. Polysaccharides/peptides/nucleic acids can be isolated from biological samples or can be synthesized using standard chemical synthetic methods. Some of the molecules provided can also be expressed recombinantly in a variety of prokaryotic and eukaryotic expression systems by constructing an expression vector appropriate to the expression system, introducing the expression vector into the expression system, and isolating the recombinantly expressed molecule.

As used herein with respect to the molecules provided herein, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e., isolated from other proteins.

It has been found that biologically active surfaces can be created with patterned biologic agent adhesion. It has also been found that the biologically active surfaces described herein can be used to affect biological processes. For example, substrates onto which hyaluronic acid, heparin, heparan sulfate, chondroitin sulfate A, chondroitin sulfate C and dermatan sulfate significantly inhibited fibronectin binding. Heparin, heparan sulfate, chondroitin sulfate A, chondroitin sulfate C and dermatan sulfate surfaces promoted cell adhesion, while hyaluronic acid surfaces inhibited it. It was also found that most glycosaminoglycan surfaces supported cell proliferation except for hyaluronic acid, heparin and chondroitin sulfate A. Interestingly, heparan sulfate and dermatan sulfate allowed for substantial cell proliferation. Biological properties were also found to be influenced by surfaces with digested glycosaminoglycans. For instance, heparinase I-digested heparin and heparinase-III heparin digested surfaces both supported cell growth, while heparinase I-digested and heparinase-III digested heparan sulfate surfaces prevented cell growth. Further, heparinase-III digested heparin and heparinase-III digested heparan sulfate surfaces were found to allow for more protein binding.

Therefore, in one aspect of the invention methods are provided whereby biological processes are influenced using the biologically active surfaces provided herein. The methods include, for example, methods for promoting or inhibiting protein or cell binding, promoting or inhibiting cell proliferation, and promoting or inhibiting bacterial or viral adhesion. Methods and compositions are also provided whereby the biologically active surface will be “patterned”, which is intended to mean that the surface comprises two or more areas that promote a different biological process. For instance, a surface may have one area to which a protein and/or cell can adhere and another area which resists adhesion. In another example, a surface can have three areas, each which promotes the adherence of a different cell or protein. In yet another example, a surface can have two areas that promote adherence of a cell or cells and an area therebetween that resists cell adherence. The biologically active surfaces provided can be also used to alter the proliferative or adhesive properties of surrounding cells or tissue.

Furthermore, the biologically active surfaces can be used to prevent contamination or food spoilage. As used herein, “preventing contamination or food spoilage” refers to any reduction in the bacterial load of a food or the inhibition of bacterial load increase over time. The term is also used to refer to any increase in the shelf-life of a food or any improvement in the taste or flavor of a food as a result of an immobilized polysaccharide surface. Therefore, “effective to prevent contamination or food spoilage” refers to a biologically active surface that can, when placed around or in contact with a food, reduce the bacterial load, inhibit its increase or prolong the shelf-life of the food. As used herein, a “food” is any substance or product for human or animal consumption. Food products include, for example, beverages, soups, breads, crackers, baked goods, meats and produce. Meats include beef, pork, poultry or seafood (e.g., fish). Produce includes fruits and vegetables.

In one embodiment where the biologically active surface is one used to prevent contamination or food spoilage, the substrate for the biologically active surface can comprise polystyrene, an erethylene-benzene containing polymer or polyvinylidene chloride. Food can be placed in contact with a food storage device. As used herein a “food storage device” is any device that can be placed in contact with a food. Contact with a food storage device refers to placement of a food into a food storage device or covering or enclosing a food with a food storage device. Food storage devices, therefore, include wraps, such as plastic wraps, sheets or films, that can cover or surround a food, or containers into which a food can be placed. A “wrap” as used herein refers to any flexible sheet or film that can be used to cover or surround a food. Wraps, therefore, include plastic wraps or paper wraps. Preferably, paper wraps are lined with a plastic. A “food container” refers to any container into which a food can be placed. In one embodiment a food container is one that can be enclosed (e.g., with a lid). Food containers include cups, bowls, tins, jugs, boxes, bags, etc. Food containers can be made of any material appropriate for contact with a food. Such materials include glass, metals, plastics, foams, etc. In some instances the materials, such as glass, metals and foams are plastic-lined. The polysaccharides can be immobilized on these materials or on the plastic lining or both. Materials for use in food containers can also include paper-based products. Preferably, such paper-based products are plastic-lined, and the polysaccharides are immobilized on the plastic lining.

The methods provided include the steps of providing a biologically active surface in an in vitro or in vivo system such that a biological process will be affected by the presence of the biologically active surface. In one embodiment the biologically active surface is provided to a subject via implantation. In another embodiment the biologically active surface affects a biological process in an in vitro system whereby a sample (e.g., a sample of cells, a subcellular preparation or components thereof) come in contact with the biologically active surface. In one embodiment the biologically active surface is used in a device to filter a sample (e.g., a liquid sample or fluid). In another embodiment the filtering device filters bacteria and/or viruses. In still another embodiment the substrates provided can be used to promote implant adhesion (e.g., orthopedic implants) or prevent infectious agents from attaching to the implant.

Also contemplated herein are methods of determining a cellular response. The cells can be obtained from a subject or can be from a cell line. These methods can include contacting the biologically active surfaces provided herein with one or more cell populations and testing the cells for the production of a protein or nucleic acid that encodes it that is correlated with a particular biological response (i.e., a marker for the response). Such methods can be used, for example, to determine the level of proliferation or adhesion by measuring the amount and/or phosphorylation state of proliferative proteins (e.g., extracellular receptor activated kinase (ERK), MAP and ERK kinase (MEK), adhesion related proteins (e.g., CD44 or focal adhesion kinase (FAK)) and apoptosis related proteins (e.g., Akt/protein kinase B (PKB), caspases, etc.) using techniques including fluorescent screens. Screening for such markers can be used, therefore, for diagnostic purposes or for identifying therapeutic agents. Therapeutic agents can be identified using the methods provided herein that are useful for a variety of therapeutic endpoints, which include treating cancer, inhibiting metastasis, treating a neurodegenerative disease, inhibiting coagulation, treating asthma, inhibiting infection, preventing the attachment of infectious agents, promoting wound healing, promoting implant adhesion, treating inflammatory bowel disease, inhibiting inflammation, promoting or inhibiting angiogenesis, etc. The methods of determining cellular response can further include treating one or more of the cell populations with an agent, such as a therapeutic agent, prior to or concomitant with contacting the cells with the biologically active surface. Methods of evaluating the effectiveness of a particular therapeutic agent, therefore, are also provided.

Also provided is a method of screening, which includes contacting a biologically active surface provided herein with a sample containing one or more cell types, a subcellular preparation or components thereof and testing for a specific response. As used herein a “specific response” includes binding, adhesion, proliferation, migration, etc. The sample can also be contacted with an agent, such as a therapeutic agent, prior to or concomitant with contacting the biologically active surface. When two or more cell populations are used the cells can be of similar or dissimilar origin. The screening methods, therefore, in some embodiments can be methods for testing or comparing two or more cell populations.

It follows, therefore, that the biologically active surfaces provided can also be used as or in medical devices. Such medical devices can be any implantable device. The medical device, for example, can be a tissue scaffold, stent, shunt, valve, pacemaker, pulse generator, cardiac defibrillator, spinal stimulator, brain stimulator, sacral nerve stimulator, lead, inducer, sensor, screw, anchor, pin, adhesion sheet, needle, lens, joint, prosthetic/orthopedic implant, catheter, tube (e.g., tubes for lines and drains), suture, etc.

Biologically active surfaces can also be created not only on medical and filtering devices but also on particles (e.g., inhalable particles, particles for oral or rectal delivery, etc.), pills and on slow release drug delivery vehicles. Such coatings can be used to prevent cell seeding, infection, fibrotic reactions, etc. For example, inhalable particles, for instance, can be coated with polysaccharides, such as heparin, hyaluronic acid, etc., to seed various parts of the airway and to prevent infection. Such particles can be used in the treatment of subjects with respiratory ailments, such as asthma and chronic obstructive pulmonary disease. The particles can also be used in the treatment of subjects with cystic fibrosis. In another example, the coated particles provided can be used in oral and rectal (e.g., as a stool loosener) delivery. In some embodiments of the invention polysaccharide coatings can be used on slow delivery vehicles (e.g., PEGylated, calcium alginate, etc. delivery vehicles) or spheres that are used to deliver drugs. In one embodiment glycosaminoglycans can be used to coat such a drug delivery device to resist binding of the delivery vehicle to proteins. In one specific embodiment the drug to be delivered is an albumin-binding drug and the glycosaminoglycan resists albumin binding. In another embodiment the drug delivery vehicle is for ocular administration.

The compositions and devices provided can be used in a variety of methods, such as methods of treatment. Methods are, therefore, provided for any treatment regimen that would benefit from the use of the biologically active surfaces provided herein. Such methods include methods for treating coagulant disorders, cancer, neurodegenerative disorders, asthma, inflammatory bowel disease, etc. The methods also include methods for preventing infection or preventing the attachment of infectious agents, inhibiting or promoting angiogenesis, preventing inflammation, promoting implant adhesion and promoting wound healing.

The invention, therefore, is useful for treating cancer (i.e., tumor cell proliferation and/or metastasis) in a subject. The terms “treat” and “treating” as used herein refer to inhibiting completely or partially the proliferation or metastasis of a cancer or tumor cell, as well as inhibiting any increase in the proliferation or metastasis of a cancer or tumor cell. Treat or treating also refers to retarding the proliferation or metastasis of tumor cells in a subject. Additionally, treat or treating may include the elimination or reduction of the symptoms associated with the tumor cell proliferation or metastasis. The medical device, therefore, comprises a biologically active surface, which contains a polysaccharide, such as those provided herein and, optionally, an additional biological or therapeutic agent, such as an anti-cancer agent. In one embodiment the medical device can be implanted near the site of a tumor. In another embodiment a coated particle, pill or delivery vehicle can be administered to a subject with cancer. In some embodiments the coated particle, pill or delivery vehicle further contains an anti-cancer agent.

A “subject having a cancer” is a subject that has detectable cancerous cells. The cancer may be a malignant or non-malignant cancer. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with the biologically active surfaces provided, alone or in combination with an additional therapeutic, the subject may be able to kill the cancer cells as they develop.

The cancer can be any cancer, including melanoma, hepatic adenocarcinoma, prostatic adenocarcinoma or osteosarcoma. Other cancers include biliary tract cancer; bladder cancer; breast cancer; brain cancer including glioblastomas and medulloblastomas; Burkitt's lymphoma, cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease; lung cancer including small cell lung cancer and non-small cell lung cancer; lymphomas including Hodgkin's disease and lymphocytic lymphomas; neuroblastomas; oral cancer including squamous cell carcinoma; esophageal cancer; ovarian cancer including those arising from epithelial cells, stromal cells, germ cells and mesenchymal cells; pancreatic cancer; rectal cancer; sarcomas including leiomyosarcoma, rhabdomyosarcoma, liposarcoma, fibrosarcoma, and synovial sarcoma; skin cancer including Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

The biologically active surfaces provided may also be used, for instance, in a method for inhibiting angiogenesis. In this method a biologically active surface as provided herein is administered (i.e., implanted) in a subject in need of treatment thereof. Angiogenesis as used herein is the formation of new blood vessels.

“Angiogenesis” often occurs in tumors when endothelial cells secrete a group of growth factors that are mitogenic for endothelium causing the elongation and proliferation of endothelial cells which results in a generation of new blood vessels. The biologically active surfaces are also useful for inhibiting neovascularization associated with disease such as eye disease. Neovascularization, or angiogenesis, is the growth and development of new arteries. It is critical to the normal development of the vascular system, including injury-repair. There are, however, conditions characterized by abnormal neovascularization, including diabetic retinopathy, neovascular glaucoma, rheumatoid arthritis, and certain cancers. For example, diabetic retinopathy is a leading cause of blindness. There are two types of diabetic retinopathy, simple and proliferative. Proliferative retinopathy is characterized by neovascularization and scarring. About one-half of those patients with proliferative retinopathy progress to blindness within about five years.

Another example of abnormal neovascularization is that associated with solid tumors. It is now established that unrestricted growth of tumors is dependant upon angiogenesis, and that induction of angiogenesis by liberation of angiogenic factors can be an important step in carcinogenesis. For example, basic fibroblast growth factor (bFGF or FGF2) is liberated by several cancer cells and plays a crucial role in cancer angiogenesis. As used herein, an angiogenic condition means a disease or undesirable medical condition having a pathology including neovascularization. Such diseases or conditions include diabetic retinopathy, neovascular glaucoma and rheumatoid arthritis (non-cancer angiogenic conditions). Cancer angiogenic conditions are solid tumors and cancers or tumors otherwise associated with neovascularization such as hemangioendotheliomas, hemangiomas and Kaposi's sarcoma.

Proliferation of endothelial and vascular smooth muscle cells is the main feature of neovascularization. Thus the substrates of the invention are useful for preventing proliferation and, therefore, inhibiting or arresting altogether the progression of the angiogenic condition which depends in whole or in part upon such neovascularization.

As provided elsewhere herein, the biologically active surfaces provided can further comprise an additional therapeutic agent. Additionally, the biologically active surfaces can be used in conjunction with separately administered therapeutic agents.

Additional therapeutic agents include anti-cancer agents. Anti-cancer agents include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazoflirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Additional agents further include agents that treat the side-effects of radiation therapy, such as anti-emetics, radiation protectants, etc.

Anti-cancer agents also can include cytotoxic agents and agents that act on tumor neovasculature. Cytotoxic agents include cytotoxic radionuclides, chemical toxins and protein toxins. The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Toxins also include poisonous lectins, plant toxins such as ricin, abrin, modeccin, botulina and diphtheria toxins. Of course, combinations of the various toxins are also provided thereby accommodating variable cytotoxicity. Other chemotherapeutic agents are known to those skilled in the art.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein), interferon inducible protein 10 (U.S. Pat. No. 5,994,292), and the like. Anticancer agents also include immunomodulators such as α-interferon, γ-interferon, and tumor necrosis factor alpha (TNFα).

The promotion of angiogenesis or neovascularization, however, can also be desirable. For example, angiogenesis would be desirable in tissue engineering applications, such as with the use of stents, prosthetic implants, skin grafts, artificial skin, vascular grafts, or any application where increased vascularization is desirable. Compositions and methods are, therefore, provided for the promotion of angiogenesis, preferably, for tissue engineering applications. In one embodiment the biologically active surface can include an angiogenic factor such as VEGF, FGF, EGF, PDGF or hepatocyte growth factor (HGF). In another embodiment the biologically active surface can include a glycosaminoglycan which promotes adhesion to the surrounding cells or tissue as well as an angiogenesis promoting factor.

The invention also contemplates the treatment of subjects having or at risk of developing a neurodegenerative disorder, such as a neurodegenerative disease or suffering an injury to nerve cells. Neuronal cells are predominantly categorized based on their local/regional synaptic connections (e.g., local circuit intemeurons vs. longrange projection neurons) and receptor sets, and associated second messenger systems. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc.

“Neurodegenerative disorder” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). and Wernicke-Korsakoff's related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

The biologically active surfaces provided herein can be combined with other therapeutic agents used to promote nerve regeneration or treat neurodegenerative disease.

For example, antiparkinsonian agents include but are not limited to Benztropine Mesylate; Biperiden; Biperiden Hydrochloride; Biperiden Lactate; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinelorane Hydrochloride; Ropinirole Hydrochloride; Selegiline Hydrochloride; Tolcapone; Trihexyphenidyl Hydrochloride. Drugs for the treatment of amyotrophic lateral sclerosis include but are not limited to Riluzole. Drugs for the treatment of Paget's disease include but are not limited to Tiludronate Disodium.

The biologically active surfaces provided are also useful for treating or preventing disorders associated with coagulation. A “disease associated with coagulation” as used herein refers to a condition characterized by inflammation resulting from an interruption in the blood supply to a tissue, which may occur due to a blockage of the blood vessel responsible for supplying blood to the tissue such as is seen for myocardial, cerebral infarction, or peripheral vascular disease, or as a result of embolism formation associated with conditions such as atrial fibrillation or deep venous thrombosis. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue. Coagulation associated diseases/states also include disseminated intravascular coagulation, venous stasis, pregnancy, cancer, hemophilia, clotting factor deficiencies, etc.

The biologically active surfaces, therefore, may also contain a therapeutic agent for treating a disease associated with coagulation or the biologically active surfaces can be used to treat a disease associated with coagulation in addition to a separately administered therapeutic agent. Examples of therapeutics useful in the treatment of diseases associated with coagulation include anticoagulation agents, antiplatelet agents, and thrombolytic agents.

Anticoagulants include, but are not limited to, heparin, modified heparins, dermatan sulfate, oversulfated dermatan sulfate, warfarin, coumadin, dicumarol, phenprocoumon, acenocoumarol, ethyl biscoumacetate, and indandione derivatives.

Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

Thrombolytic agents include, but are not limited to, plasminogen, a₂-antiplasmin, streptokinase, antistreplase, tissue plasminogen activator (tPA), and urokinase.

Additional agents for the inhibition of coagulation include clotting factors and antithrombins, such as antithrombin 3.

In addition, as the surfaces provided are able to modulate bacterial and/or viral adhesion, the surfaces provided can be used to prevent infection or to prevent the attachment of infectious agents to a medical device. As used herein to “prevent infection” refers to the inhibition of the proliferation or survival of an infectious agent, such as bacteria and/or viruses, or to the reduction of the symptoms associated with infection. The substrates provided can be used to prevent urinary tract infection, post-surgical wound infection, etc. The surfaces provided, therefore, can also include in some embodiments other anti-infective agents. Anti-infective agents include, for example, Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro), Alcohol; Aminacrine Hydrochloride; Benzethonium Chloride : Bithionolate Sodium; Bromchlorenone; Carbamide Peroxide; Cetalkonium Chloride; Cetylpyridinium Chloride : Chlorhexidine Hydrochloride; Clioquinol; Domiphen Bromide; Fenticlor; Fludazonium Chloride; Fuchsin, Basic; Furazolidone ; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal : and Troclosene Potassium.

Similarly, the surfaces provided can promote wound healing. Therefore, the surfaces can also, optionally, contain wound healing agents, which include, collagen to increase wound strength and promote platelet aggregation and fibrin formation; growth factors, such as platelet-derived growth factor, platelet factor 4, transforming growth factor-β; tissue factor VIIa, thrombin, fibrin, plasminogen-activator initiator, adenosine diphosphate, etc.

Additionally, the surfaces provided can also, optionally, include anti-inflammatory agents, which include Alclofenac; Alclometasone Dipropionate; Algestone Acetonide; Alpha Amylase; Amcinafal; Amcinafide; Amfenac Sodium; Amiprilose Hydrochloride; Anakinra; Anirolac ; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab ; Enolicam Sodium ; Epirizole ; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Additional agents that can also be included in the compositions provided include glycosaminoglycan-degrading enzymes and glycosaminoglycan binding proteins (e.g., EGF, VEGF, PDGF, FGF, etc.).

Effective amounts of the therapeutic agents are administered to subjects in need of such treatment. The therapeutic agents can be the immobilized polysaccharides, the other biologic or therapeutic agents provided on the biologically active surfaces, the separately administered therapeutics or some combination thereof. Effective amounts are those amounts which will result in the desired therapeutic endpoint, such as the reduction in cellular proliferation or metastasis, the promotion or inhibition of adhesion, etc., without causing other medically unacceptable side effects. An effective amount can refer to the amount of one therapeutic agent for achieving the desired therapeutic endpoint. However, in some embodiments an effective amount refers to the amount of a combination of therapeutic agents that achieves the desired therapeutic endpoint. In these embodiments it is, therefore, possible that the amount of the therapeutic agents individually is not effective to achieve the therapeutic endpoint, while their combination is.

Effective amounts can be determined with no more than routine experimentation. It is believed that doses ranging from 1 nanogram/kilogram to 100 milligrams/kilogram, depending upon the mode of administration, will be effective. The absolute amount will depend upon a variety of factors (including whether the administration is in conjunction with other methods of treatment, the number of doses and individual patient parameters including age, physical condition, size and weight) and can be determined with routine experimentation. It is preferred generally that a maximum dose be used, that is, the highest safe dose according to sound medical judgment.

In some aspects of the invention the effective amount is that amount effective to prevent invasion of a tumor cell across a barrier. The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

In some embodiments, effective amounts are those that can be used for promoting nerve regeneration. A subject in need of such treatment includes subjects that suffer from nerve disorders, such as diseases associated with neurodegeneration and injuries that result in nerve damage, in which nerve regeneration is desirable. In some embodiments the subject suffers from a central nervous system injury, such as a spinal cord injury. The effective amount can partially or completely promote nerve cell regeneration and/or motility or migration of a nerve cell. Effective amount for this type of treatment also refer to partially or completely restoring motor/physical function and/or axon regeneration. The nerve cells may be treated in vivo, in vitro, or ex vivo. Thus, the cells may be in an intact subject or isolated from a subject or alternatively may be an in vitro cell line.

A subject is any human or non-human vertebrate, e.g., dog, cat, horse, cow, pig.

Kits comprising the surfaces and compositions discussed herein are also provided. The kits can further include diagnostic agents, such as labels or an additional therapeutic agent.

In general, when administered for therapeutic purposes, the medical devices of the invention are applied in pharmaceutically acceptable form.

In other embodiments the medical devices/substrates provided are sterile.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients. The formulations can also be sterile.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, with one or more pharmaceutically acceptable carriers and optionally other therapeutic ingredients. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with each other, in a manner such that there is no interaction which would substantially impair the desired pharmaceutical efficiency.

A variety of administration routes are available. The particular mode selected will depend, of course, upon the particular active agent selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. A preferred mode of administration is a parenteral route. The term “parenteral” includes subcutaneous injections, intravenous, intramuscular, intraperitoneal, intrasternal injection or infusion techniques. Other modes of administration include oral, mucosal, rectal, vaginal, sublingual, intranasal, intratracheal, inhalation, ocular, transdermal, etc.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may conveniently be presented in unit dosage form and may be prepared by any of the methods well known in the art of pharmacy.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. Nos. 4,748,034 and 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

In other embodiments methods and compositions are provided whereby blood vessels or medical devices can be coated with glycosaminoglycans in vivo, for instance, by the administration of one or more glycosaminoglycans to the bloodstream or by localized administration at a time separate from the administration of the medical device. In one embodiment the device is implanted with or without an immobilized glycosaminoglycan prior to the administration of a glycosaminoglycan. The glycosaminoglycan can be administered in an amount and in a way (e.g., to the bloodstream) such that it is immobilized on the surface of the device. In one embodiment the amount of the ultimately immobilized glycosaminoglycan is an amount effective to affect a biological process. In another embodiment a glycosaminoglycan is attached to a blood vessel or device by binding to another agent, such as another polysaccharide, which is administered prior to or concomitantly with the glycosaminoglycan. In this embodiment the agent binds the device or blood vessel and the glycosaminoglycan subsequently binds to the agent such that it is immobilized. Preferably the glycosaminoglycan that binds is in an amount effective for a particular therapeutic endpoint. In one embodiment the agent is a polycation.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Hyaluronic Acid Directly Immobilized on Solid Substrates

HA has received much attention due to its unique properties. HA is a linear polysaccharide composed of repeating disaccharide units of N-acetyl-D-glucosamine linked to D-glucuronic acid, and unlike all other GAGs, HA is not sulfated. As a component of the extracellular matrix, HA plays an important role in lubrication, water-sorption, water-retention and a number of cellular functions such as attachment, migration and proliferation.^{12, 13} HA, therefore, can be a building block for new biocompatible and biodegradable polymers that have applications in drug delivery, tissue engineering and viscosupplementation.¹⁴⁻¹⁷

The formation of a stable HA coating has potential applications ranging from bioactive surfaces to the formation of multilayer polyelectrolyte films.¹⁸⁻²⁰ To generate HA-coated surfaces various immobilization techniques have been employed ranging from covalent attachment,^{9 21-23} layer-by-layer deposition^{24,25} and binding with natural ligands such as p32²⁶. These strategies, however, involve potentially complicated synthetic approaches that require the use of chemicals, ultraviolet (UV) light or cumbersome procedures to prepare additional binding layers, potentially limiting their application as a general route to HA surface immobilization.

Here, the formation of a stable, chemisorbed HA layer on hydrophilic surfaces, such as glass and silicon oxides, is demonstrated and characterized using X-ray photoelectron microscopy (XPS), ellipsometry and atomic force microscopy (AFM). In addition, the underlying mechanism, by studying HA layer formation at various pH conditions and with washing procedures, was examined. Evidence suggests that the HA is stabilized on the surface through hydrogen bonding between the hydrophilic moieties in HA, such as carboxylic acid (—COOH) or hydroxyl (—OH) groups with silanol (—SiOH), carboxylic acid or hydroxyl groups on the hydrophilic substrates. The chemisorbed HA layer remains stable in phosphate buffered saline (PBS) for at least 7 days without losing its resistant properties. This behavior is related to the molecular entanglement and intrinsic stiffness of HA as a result of strong internal and external hydrogen bonding as well as high molecular weight. HA is a biological molecule that can be directly immobilized on substrates with high efficiency and stability.

Materials And Methods

Materials

HA (lot # 904572, Mₙ=2.1 MDa by light scattering) was kindly supplied by Genzyme Inc. (Boston, Mass.). Silicon dioxide wafers (1 μm of SiO₂ on Si) were purchased from International Wafer Service (Portola Valley, Calif.) and used without further treatment. Heparin and heparan sulfate were from Celsus Laboratories (Columbus, Ohio). Chondroitin sulfate A, chondroitin sulfate C, dermatan sulfate, fluorescein isothiocyanate-labeled bovine serum albumin (FITC-BSA), goat anti-rabbit immunoglobulin G (FITC-IgG), fibronectin (FN) and anti-FN antibody were purchased from Sigma (St. Louis, Mo.). Glass slides were treated with O₂ plasma for 1 min to generate —OH groups as well as to clean the surfaces unless otherwise indicated.

Surface Characterization

Fluorescent optical images were obtained using an inverted microscope (Axiovert 200, Carl Zeiss AG, Thornwood, N.Y.). XPS spectra were recorded using a Kratos AXIS Ultra spectrometer (Kratos Analytical, Inc., Chestnut Ridge, N.Y.). Spectra were obtained with a monochromatic Al K_{α} X-ray source (1486.6 eV, Kratos Analytical, Inc.). Pass energy was 160 eV for survey spectra and 10 eV for high-resolution spectra. All spectra were calibrated with reference to the unfunctionalized aliphatic carbon at a binding energy of 285.0 eV. Spectra were recorded with similar settings (number of sweeps, integration times, etc.) from sample to sample to enable comparisons to be made. The analysis of the XPS spectra was performed on the basis of 90° unless otherwise indicated. Atomic force micrographs were obtained with tapping mode on a NanoScope III Dimension (Veeco Instruments, Rochester, N.Y.) in air. The scan rate was 0.5 Hz and 256 lines were scanned per sample. Tapping mode tips, NSC15-300 kHz, were obtained from MikroMasch (Portland, Oreg.). Data were processed using Nanoscope III 4.31r6 software (Veeco Instruments Inc.). The thickness of the chemisorbed HA layer was measured with a Gaertner L116A ellipsometer (Gaertner Scientific Corp., Skokie, Ill.) with a 632.8 nm He—Ne laser. A refractive index of 1.46 was used for all HA films, and a three-phase model was used to calculate thicknesses.

Construction and Stability of a Chemisorbed Layer and Testing Protein Adsorption

A few drops of HA solution (5 mg/mL in distilled water) were placed on the surface and spin-coated (Model CB 15, Headway Research, Inc., Garland, Tex.) at 1000 rpm for 10 s. The samples were stored overnight at room temperature to allow the solvent to evaporate. To examine the effect of washing, some samples were washed several times within 30 min of spin coating and then dried with a mild nitrogen stream. To examine the effect of pH, the silicon oxide surfaces were exposed for several hours to solutions of pH 2, 7, and 11 (HCl and NaOH mixtures), respectively, leading to different oxidization states. HA films were prepared on those surfaces using the same procedure described above. In addition to HA, thin films of the other polysaccharides were prepared in the same manner.

To measure the immobilization of HA, heparin, HS, CS A, CS C and DS films, fluorescent staining for adhesion of various proteins on the coated surfaces was performed. FITC-labeled BSA (50 μg/mL), IgG (50 μg/mL) and FN (20 μg/mL) were dissolved in PBS solution (pH=7.4; 10 mM sodium phosphate buffer, 2.7 mM KCl, and 137 mM NaCl). To measure FN adsorption, the surfaces were stained with anti-FN antibody for 45 min, followed by a 1 h incubation with the FITC-labeled anti-rabbit secondary antibody. A few drops of the protein solution were evenly distributed onto the HA surfaces. After storing at room temperature for 30 min, the surfaces were rinsed with PBS solution and water and then blown dry in a stream of nitrogen. To analyze stability, HA surfaces were placed in a PBS bath at various times and stored at room temperature for up to 7 days. The PBS solution was changed daily to prevent readsorption of dissociated HA onto the surface. The stability was subsequently analyzed by testing for FN adsorption. The slides were then examined under a fluorescent microscope under a UV light exposure of 2 seconds. Blank glass slides with or without FN staining were used as positive and negative controls, respectively. The fluorescent images were analyzed quantitatively using Scion Image (Scion Corporation, Frederick, Miss.), and the statistical analysis was performed using one-sided ANOVA tests with p<0.05 to distinguish statistical significance.

Results

Detection of a Chemisorbed HA Layer

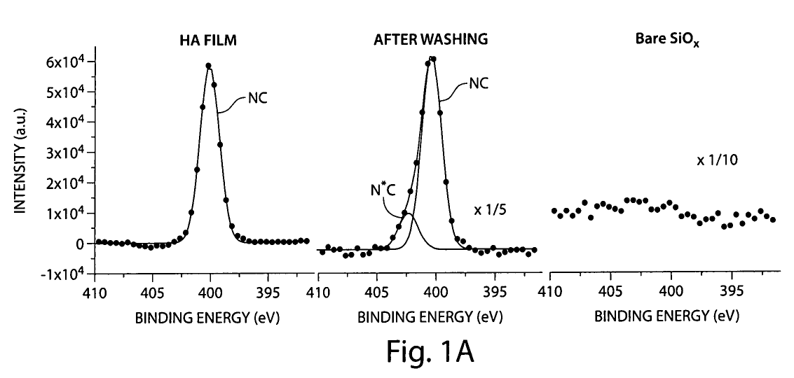

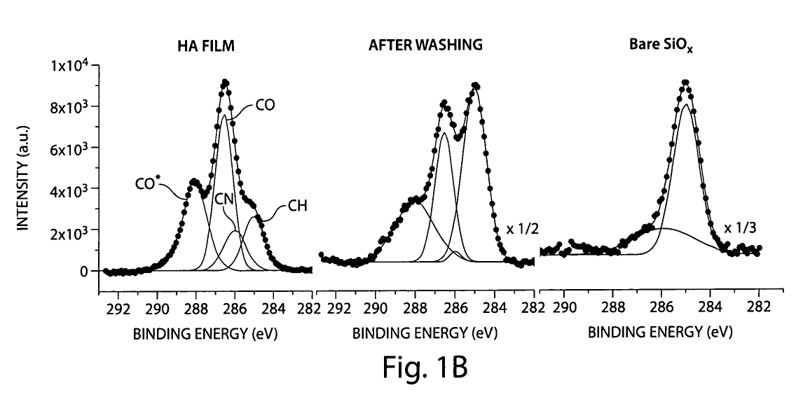

The presence of a chemisorbed HA layer on silicon dioxide substrates or glass was verified by analyzing the elemental composition (carbon, oxygen, nitrogen, and silicon) of the surfaces using XPS. In particular, the detection of nitrogen in the XPS spectra was strong evidence to support the presence of a residual HA layer (FIG. 1) since nitrogen is found in HA but not the substrate. As expected, no nitrogen was detected on the bare silicon oxide. The intensity at 400.1 eV (N 1s) decreased to about 25% of its original intensity after washing with PBS, though the peak remained, indicating a residual layer of HA (FIG. 1A). A new XPS peak was also detected at 402.3 eV (15.5%) after washing, suggesting a modified oxidation state of nitrogen, denoted N*C. It was hypothesized that the new peak originates from the partial protonation or hydrogen bonding of nitrogen to silanol groups (—SiOH) on the surface. The persistence of the nitrogen peak and the emergence of a new oxidized state (N*C) generated after washing are consistent with a residual layer on the surface formed by chemical interactions between the layer and the substrate.

The carbon peak (C 1s) of an as-spun film contains four peaks that are located at 285.0 (16.1%), 286.1 (12.7%), 286.6 (40.0%) and 288.1 (31.2%), consistent with previous reports (FIG. 1B).²⁷ The amount of unfunctionalized hydrocarbon (285 eV) was higher than expected (7.1%),²⁷ which may be attributed to carbon adsorption from the air. In order of increasing binding energies, these peaks represent the hydrocarbon environment (HC), carbon singly bound to nitrogen (CN), carbon singly bound to oxygen (CO), strongly oxidized carbons (CO*) including carbon doubly bound to oxygen and a combined peak representing both amide and carboxylate ion carbon atoms (CON and COO).²⁷ In contrast to the as-spun coatings, the relative intensities were substantially changed after washing with the peak locations slightly shifted. Two factors potentially responsible for this behavior are the increased portion of unfunctionalized hydrocarbon from the substrate, and the surface interactions between HA and the substrate. Based on the modified oxidation state of nitrogen in the XPS spectra and hydrophilic moieties in HA some strong interactions, such as hydrogen bonding, are believed to play a role in the formation of the chemisorbed layer. In a separate experiment, the HA film was completely washed away on hydrophobic substrates such as untreated polystyrene, which indicates that other hydrophobic interactions could be ruled out in examining the origin of the chemisorbed layer.

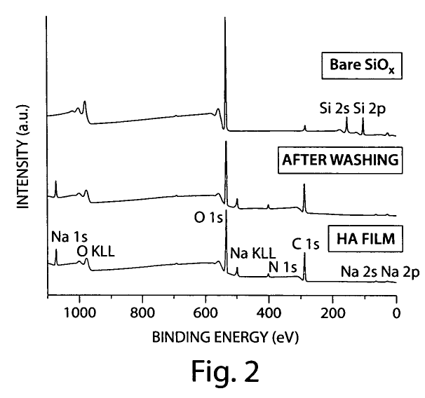

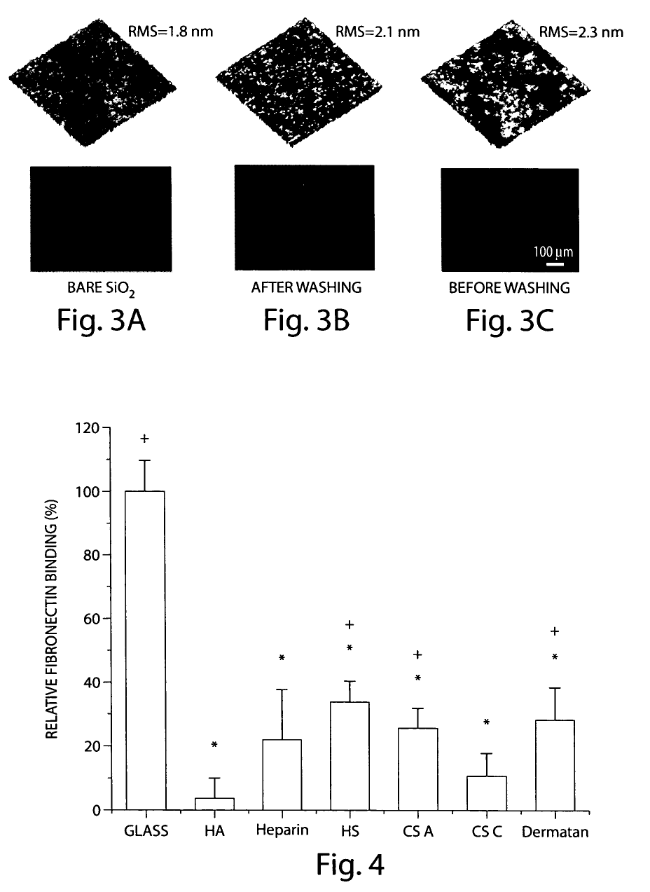

To analyze the thickness of the HA film, ellipsometry, AFM and XPS measurements at two different angles were used. At a 90° take-off angle (long penetration depth), silicon peaks were not seen for an as-spun sample (i.e., thick HA film on a glass), as opposed to bare silicon oxide and washed film controls. On the other hand, silicon peaks were nearly absent on the washed film when a 30° take-off angle was used (short penetration depth) (FIG. 2). This indicates that the residual film was extremely thin, less than 5-10 nm depending on the element and electron selected, the thickness ranging within the penetration depth and the substrate surface was nearly fully covered with the chemisorbed layer. The presence of the chemisorbed layer was further confirmed by ellipsometry and AFM measurements. The ellipsometry results indicated that the initial thickness of the HA film was about 330 nm, which decreased drastically to about 3 nm after washing and then remained at the same value. Furthermore, the roughness of a residual layer (2.1 nm) was between that of the substrate (1.8 nn) and the as-spun film (2.3 nm), which also supports the presence of a residual layer (FIG. 3).

To further explore the potential mechanism of adhesion, silicon oxide surfaces were exposed to three different pH values of 2, 7, and 11 to test the effects of surface charge and hydrophobicity on the formation of a HA coating. At acidic conditions (pH=2), the hydroxyl groups present on the surface are protonated (OH₂⁺) such that the adsorption of HA should be enhanced due to negative charge of HA. In contrast, since the surface is negatively charged (O⁻), the adsorption would be reduced at basic conditions (pH=11). At pH 11, the atomic mass percentage of nitrogen on the surface was 0.33% whereas it increased to 3.61% when exposed to pH 2 (Table 1). These results indicate that HA is more likely to adsorb to positively charged surfaces than negatively charged surfaces. Interestingly, neutral surfaces (pH=7) were also effective in adhering HA (3.34%), which also supports the presence of hydrogen bonding between HA and the hydroxyl groups.

TABLE 1
Atomic Mass Percentage of Carbon, Nitrogen, Oxygen And Silicon
Elements for HA Films Formed Under Various Conditions

Atomic conc. %

| Sample | C | N | O | Si |
|---|---|---|---|---|
| Exposure to pH 2 | 57.6 | 3.6 | 34.0 | 4.8 |
| Exposure to pH 7 | 52.2 | 3.3 | 38.3 | 6.2 |
| Exposure to pH 11 | 14.6 | 0.3 | 58.0 | 27.1 |
| No washing + drying | 49.2 | 2.7 | 38.8 | 9.3 |
| Washing after 30 min + drying | 11.7 | 0.7 | 64.6 | 23.0 |
| Bare silicon dioxide | 4.2 | 0 | 65.4 | 30.4 |

Errors are within 5%.

Whether the current approach is ubiquitous in immobilizing polymers having hydrophilic moieties on hydrophilic substrates was explored. A previous study reported that carboxyl (—COOH) groups were confined onto hydrophilic surfaces with additional thermal polymerization.²⁸ Poly(ethylene glycol)s, however, detach from the substrates upon hydration despite having hydrophilic moieties (—OH). It was hypothesized that two factors contribute to the formation of a chemisorbed HA layer. The first is hydrogen bonding strong enough to endure the polymer swelling stress at the interface upon exposure to water. The second is a dense molecular structure, such as entanglement, to prevent penetration of water molecules of the chemisorbed layer. Thus, sufficiently strong hydrogen bonding is required to prevent the adsorbed layer from peeling off from the surface. In this regard, the HA film should have enough contact time with the surface to build a robust interface. As indicated by XPS, the amount of nitrogen adsorbed onto the surface was lower when the sample was washed within 30 min after spin coating (0.69%) (Table 1) and significantly increased to 2.74% when the sample was dried overnight prior to washing. This indicates that the duration of exposure and sample drying play a role in the adsorption of the HA onto the surfaces.

With respect to the density of the molecular structure, HA is a highly hydrated polyanion, which forms a network between domains in solutions.^{29, 30} In addition, the polymer shows intrinsic stiffness due to hydrogen bonds between adjacent saccharides. HA is immobilized on silicon and other dioxide surfaces in higher quantities than other polysaccharides including dextran sulfate, heparin, HS, chondroitin sulfate, DS and alginic acid (Table 2) based on the highest nitrogen composition (3.75%) and the lowest oxygen to carbon ratio (0.64%). This behavior could be attributed to either intrinsic differences between the molecular structures of various polysaccharides or their lower molecular weights compared to HA.

TABLE 2
Atomic Mass Percentage of GAG Surfaces and Control Surfaces

| Sample | N | O | C | O:C |
|---|---|---|---|---|
| Untreated | 0.00 | 92.4 | 7.6 | 12.2 |
| HA | 3.8 | 37.5 | 58.7 | 0.6 |
| Heparin | 0.2 | 89.4 | 10.4 | 8.6 |
| HS | 0.1 | 91.1 | 8.8 | 10.4 |
| CS A | 0.5 | 88.8 | 10.7 | 8.3 |
| CS C | 0.1 | 90.5 | 9.4 | 9.6 |
| DS | 0.4 | 89.0 | 10.6 | 8.4 |

XPS was performed on GAG surfaces formed on silicon dioxide after washing. Untreated surfaces are silicon dioxide only. Numbers for nitrogen, oxygen, and carbon refer to atomic mass percentage. Oxygen:Carbon (O:C) is the atomic mass percentage of oxygen divided that by carbon. Errors are within 5%.

Protein Resistance, Degradability and Stability of a Chemisorbed HA Layer

To test the effectiveness of the HA surfaces for protein resistance, HA modified surfaces were exposed to FITC-BSA, FITC-IgG and FN. The adhesion of BSA (0.46%), IgG (7.81%) and FN (6.22%) was significantly reduced (p<0.001) on HA-coated surfaces compared to glass controls (100%) as measured by fluorescence intensity. A typical example of the fluorescent images for a bare silicon oxide, a HA surface after thorough washing, and an as-coated HA film is shown in FIG. 3 when FN is applied to the surface with subsequent antibody staining. As seen from the figure, HA is uniformly attached to the surface even after extensive washing. Protein resistance of various other polysaccharide surfaces on glass was also tested using FN (FIG. 4). Surfaces formed with other polysaccharides resisted the adsorption of FN significantly higher than glass controls (p<0.05). Despite this, most other polysaccharide surfaces were still less resistant to FN absorption than HA coatings (p<0.05).

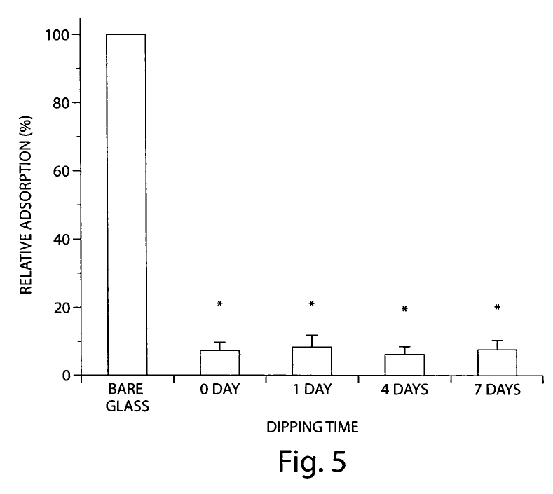

Although HA is biodegradable in nature, the possibility of degradation can presumably be ruled out herein since oxidants such as HO* and HOC/ClO⁻ are believed to be important in the degradation of HA. The generation of reactive oxygen species is mediated by metal-ion catalysis (HO⁻) in vitro^{31,32} or myeloperoxidase catalyzed reaction of H₂O₂ with Cl⁻ (HOCl/ClO⁻) in vivo. To investigate long-term stability, XPS was performed on the aged samples, which revealed persistent nitrogen peaks even after a week in PBS solution. However, the uniform distribution of HA is difficult to measure by means of XPS. Therefore, fluorescent staining of the samples as a fumction of time was used to obtain a global assessment of HA adsorption. The chemisorbed HA layer was also stable for at least 7 days as determined by the analysis of fluorescent images (FIG. 5). The presence of the HA surface greatly reduced the adsorption of FN (>92%), even after the surface was exposed to PBS for 7 days prior to exposure, FN adsorption and staining. These results indicate that, at least in the case of silicon dioxide, the formation of a chemisorbed layer of HA is stable for at least one week.

Despite the water solubility and hydrophilic nature of HA, HA can be directly immobilized onto glass and silicon oxide substrates because of hydrogen bonding and high molecular weight. An ultrathin HA layer of about 3 nm is left behind even after extensive washing with PBS or water. The presence of this layer was verified with XPS, elliposometry and AFM measurements. Fluorescent staining and XPS showed that the resulting surfaces remain stable for at least 7 days. Thus, the approach is a general route to the immobilization of HA and provides a new way to attach other bioactive molecules having hydrophilic moieties to solid substrates.

### References for Example 1

1. Morra M. On the molecular basis of fouling resistance. J Biomater Sci Polym Ed 2000; 11:547-569.
2. Piehler J, Brecht A, Hehl K, Gauglitz G. Protein interactions in covalently attached dextran layers. Colloids and Surfaces B-Biointerfaces 1999;13:325-336.
3. Osterberg E, Bergstrom K, Holmberg K, Riggs J A, Vanalstine J M, Schuman T P, Burns N L, Harris J M. Comparison of Polysaccharide and Poly(Ethylene Glycol) Coatings for Reduction of Protein Adsorption on Polystyrene Surfaces. Colloids and Surfaces a-Physicochemical and Engineering Aspects 1993;77:159-169.
4. Osterberg E, Bergstrom K, Holmberg K, Schuman T P, Riggs J A, Burns N L, Van Alstine J M, Harris J M. Protein-rejecting ability of surface-bound dextran in end-on and side-on configurations: comparison to PEG. J Biomed Mater Res 1995;29:741-747.
5. Wang D, Liu S, Trummer B J, Deng C, Wang A. Carbohydrate microarrays for the recognition of cross-reactive molecular markers of microbes and host cells. Nat Biotechnol 2002;20:275-281.
6. Lofas S, Johnsson B. A Novel Hydrogel Matrix on Gold Surfaces in Surface-Plasmon Resonance Sensors for Fast and Efficient Covalent Immobilization of Ligands. Journal of the Chemical Society-Chemical Communications 1990:1526-1528.
7. Dai L, Zientek P, St Johns H, Pasic P, Chatelier R, Griesser H J. Surface modification of polymeric biomaterials, in Ratner B, Castner D (eds): Surface modification of polymeric biomaterials. New York, Plenum Press; 1996, p 147.
8. Hartley P G, McArthur S L, McLean K M, Griesser H J. Physicochemical properties of polysaccharide coatings based on grafted multilayer assemblies. Langmuir 2002;18:2483-2494.
9. Morra M, Cassineli C. Non-fouling properties of polysaccharide-coated surfaces. J Biomater Sci Polym Ed 1999; 10:1 107-1124.
10. Morra M, Cassinelli C, Pavesio A, Renier D. Atomic force microscopy evaluation of aqueous interfaces of immobilized hyaluronan. Journal of Colloid and Interface Science 2003;259:236-243.
11. Yoshioka T, Tsuru K, Hayakawa S, Osaka A. Preparation of alginic acid layers on stainless-steel substrates for biomedical applications. Biomaterials 2003;24:2889-2894.
12. Bulpitt P, Aeschlimann D. New strategy for chemical modification of hyaluronic acid: preparation of functionalized derivatives and their use in the formation of novel biocompatible hydrogels. J Biomed Mater Res 1999;47:152-169.
13. Oerther S, Le Gall H, Payan E, Lapicque F, Presle N, Hubert P, Dexheimer J, Netter P. Hyaluronate-alginate gel as a novel biomaterial: mechanical properties and formation mechanism. Biotechnol Bioeng 1999;63:206-215.
14. Abantangelo G, Weigel P. New frontiers in medical science: redefining hyaluronan. Amsterdam, Elsevier; 2000.
15. Balazs E A, Denlinger J L. Clinical uses of hyaluronan: the biology of hyaluronan, in Evered D, Welan J (eds): Clinical uses of hyaluronan: the biology of hyaluronan. New York, Wiley; 1989, pp 265-280.
16. Piacquadio D, Jarcho M, Goltz R. Evaluation of hylan b gel as a soft-tissue augmentation implant material. J Am Acad Dermatol 1997;36:544-549.
17. Pei M, Solchaga L A, Seidel J, Zeng L, Vunjak-Novakovic G, Caplan A I, Freed L E. Bioreactors mediate the effectiveness of tissue engineering scaffolds. Faseb J 2002;16:1691-1694.
18. Bernard B A, Newton S A, Olden K. Effect of size and location of the oligosaccharide chain on protease degradation of bovine pancreatic ribonuclease. J Biol Chem 1983;258:12198-12202.
19. Lohmander L S, De Luca S, Nilsson B, Hascall V C, Caputo C B, Kimura J H, Heinegard D. Oligosaccharides on proteoglycans from the swarm rat chondrosarcoma. J Biol Chem 1980;255 :6084-6091.
20. Miyake K, Underhill C B, Lesley J, Kincade P W. Hyaluronate can function as a cell adhesion molecule and CD44 participates in hyaluronate recognition. J Exp Med 1990;172:69-75.
21. Mason M, Vercruysse K P, Kirker K R, Frisch R, Marecak D M, Prestwich G D, Pitt W G. Attachment of hyaluronic acid to polypropylene, polystyrene, and polytetrafluoroethylene. Biomaterials 2000;21:31-36.
22. Stile R A, Barber T A, Castner D G, Healy K E. Sequential robust design methodology and X-ray photoelectron spectroscopy to analyze the grafting of hyaluronic acid to glass substrates. J Biomed Mater Res 2002;61:391-398.
23. Chen G, Ito Y, Imanishi Y, Magnani A, Lamponi S, Barbucci R. Photoimmobilization of sulfated hyaluronic acid for antithrombogenicity. Bioconjug Chem 1997;8:730-734.
24. Thierry B, Winnik F M, Merhi Y, Tabrizian M. Nanocoatings onto arteries via layer-by-layer deposition: toward the in vivo repair of damaged blood vessels. J Am Chem Soc 2003; 125:7494-7495.
25. Picart C, Lavalle P, Hubert P, Cuisinier F J G, Decher G, Schaaf P, Voegel J C. Buildup mechanism for poly(L-lysine)/hyaluronic acid films onto a solid surface. Langmuir 2001;17:7414-7424.
26. Sengupta K, Schilling J, Marx S, Markus F, Sackmann E. Supported membrane coupled ultra-thin layer of hyaluronic acid: viscoelastic properties of a tissue-surface mimetic system. Biophysical Journal 2003;84:381a-381a.
27. Shard A G, Davies M C, Tendler S J B, Bennedetti L, Purbrick M D, Paul A J, Beamson G. X-ray photoelectron spectroscopy and time-of-flight SIMS investigations of hyaluronic acid derivatives. Langmuir 1997;13:2808-2814.
28. Shibasaki Y, Seki A, Takeishi N. Thermoanalytical Study on Anchoring Effects of Long-Chain Diynoic Acids in Thermal Polymerization. Thermochimica Acta 1995;253:103-110.
29. Gribbon P, Heng B C, Hardingham T E. The molecular basis of the solution properties of hyaluronan investigated by confocal fluorescence recovery after photobleaching. Biophysical Journal 1999;77:2210-2216.
30. Kobayashi Y, Okamoto A, Nishinari K. Viscoelasticity of Hyaluronic-Acid with Different Molecular-Weights. Biorheology 1994;31:235-244.
31. Hawkins C L, Davies M J. Degradation of hyaluronic acid, poly- and monosaccharides and model compounds by hypochlorite: Evidence for radical intermediates and fragmentation. Free Radical Biology and Medicine 1998;24:1396-1410.
32. Miller R A, Britigan B E. The formation and biologic significance of phagocyte-derived oxidants. Journal of Investigative Medicine 1995;43:39-49.

### Example 2

### Glvcosaminoglycan Surfaces and the Regulation of Cell Function

Materials and Methods

Proteins and Reagents

HA (lot # 904572, Mₙ=2.1 MDa by light scattering) was generously provided by Genzyme, Inc. Silicon dioxide wafers (1 μm of SiO₂ on Si) were from International Wafer Service. Heparin and HS were from Celsus Laboratories. CS A, CS C and DS were from Sigma. Recombinant heparinases were produced as described⁵. Fetal bovine serum (FBS) was from Hyclone (Logan, Utah). L-glutamine, penicillin/streptomycin and PBS were obtained from GibcoBRL (Gaithersberg, Md.). Fluorescein isothiocyanate-labeled bovine serum albumin, fibronectin, rabbit anti-FN and goat anti-rabbit-FITC were from Sigma Chemical Co.

Production and Characterization of GAG Surfaces

Glass slides were treated with O₂ plasma for 1 minute to clean the surfaces and to generate —OH groups. Silicon dioxide wafers were not treated prior to use. Chemisorbed layers of various GAGs on solid substrates were generated as described for HA. Briefly, a few drops of 5 mg/ml solutions of various GAGs in distilled water were placed on silicon dioxide, glass or polystyrene substrates, and the films were coated by spin coating at 1000 rpm for 10 seconds. Surfaces were created with HA, heparin, HS, CS A, CS C and DS, as well as heparin and HS pretreated with hepI or hepIII. For surfaces with digested HSGAGs, heparin and HS at 5 mg/ml were treated with hepI or hepIII for 30 minutes and boiled for 30 minutes⁴. Partial digestion was confirmed by UV spectroscopy at 232 nm⁶. Once the films were cast, solvent was evaporated overnight.

Analysis of all GAG surfaces was performed after washing. To confirm GAG deposition, XPS spectra were obtained using a Kratos AXIS Ultra spectrometer, with a monochromatic Al K_{α} X-ray source (1486.6 eV). Pass energy was 160 eV for survey spectra and 10 eV for high-resolution spectra. Spectra were calibrated with respect to the unfunctionalized aliphatic carbon with a binding energy of 285.0 eV. Identical settings were used for all samples to allow for comparisons to be made. Analysis was performed at a 90° take-off angle.

The chemical and physical properties were examined by determining the contact angle of water as well as the thickness of the GAG layer. The thickness of the adsorbed GAG layers were assessed with a Gaertner L116A ellipsometer with a 632.8 nm He—Ne laser. Thickness was calculated with a three-phase model.

Protein Adsorption and Surface Stability

To measure the ability of various GAG surfaces to promote or resist protein binding, FITC-BSA and FN were dissolved in PBS (pH 7.4; 10 mM sodium phosphate buffer, 2.7 mM KCl and 137 mM NaCl) at 50 μg/ml and 20 μg/ml, respectively. Solutions were evenly distributed across the surfaces and incubated for 30 minutes. Surfaces were rinsed with PBS and dried using a stream of nitrogen gas. Surfaces on which FN was deposited were treated with anti-FN for 45 minutes and subsequently with FITC-labeled anti-rabbit secondary antibody for 60 minutes. Surfaces on which FITC-BSA was deposited were incubated 60 minutes and subsequently rinsed. The protein adhered to surfaces was imaged using an inverted microscope (Axiovert 200, Carl Zeiss AG) under a UV light exposure of 2 seconds. Blank glass slides with or without FN staining were used as positive and negative controls, respectively. The fluorescent images were analyzed quantitatively using Scion Image. Protein adhesion was quantified by normalizing the experimental case based on its relative signal intensity compared to those of the controls using the equation (Equation 1):
Percent bound=(experimental—glass slide)/(FN treated glass slide−glass slide) Equation 1

Surface stability was analyzed by establishing whether the protein adhesive properties remained. The various GAG surfaces were placed in a PBS bath and stored at room temperature for up to 4 days. The PBS solution was changed daily to prevent GAG readsorption. FN adsorption was examined for GAG surfaces stored in PBS for 1, 2, 3, and 4 days as described. Stability was assessed by determining whether the percent of protein bound remained consistent over time.

Cell Culture

B16-F10 cells (American Type Culture Collection, Manassas, Va.) were maintained in minimal essential medium (GibcoBRL) supplemented with 100 μg/ml penicillin, 100 U/ml streptomycin, 500 μg/ml L-glutamine and 10% FBS. Cells were grown in 75 cm² flasks at 37° C. in a 5% CO₂ humidified incubator. Confluent cultures were split 1:10 three times per week.

B16F10 Proliferation Assay with Free GAGs

B16F10 cultures were grown until confluent, washed with 20 ml PBS, trypsin treated (3 ml trypsin-EDTA at 37° C. for 3-5 minutes until cells detached) and pelletted (centrifuged for 3 minutes at 195×g). The supernatant was aspirated and the cells were resuspended in 10 μl proliferation media. Cell density was measured by an electronic cell counter, and the suspension was diluted to 5×10⁴ cells/ml and added to 24-well plates (1 ml/well). The cells were incubated 24 hours, serum-starved for 24 hours and treated with GAGs at final concentrations of 500 ng/ml, 5 μg/ml, 50 μg/ml and 500 μg/ml. Control cells were treated with an equivalent volume (10 μl) PBS. For experiments with digested HSGAGs, heparin and HS at 5 mg/ml in PBS were treated with hepI, hepIII or PBS for 30 minutes and boiled for 30 minutes⁴. Partial digestion was confirmed by UV spectroscopy at 232 nm⁶. Whole cell numbers were determined using an electronic cell counter after 72 hours. To determine whole cell number, cells were washed twice with PBS and treated with 500 μl/well trypsin for 5 minutes. A volume of 400 μl was removed from wells for cell counting. Average whole cell counts for experimental conditions were normalized as the percentage of control cells present at the experimental endpoint.

Cell Adhesion and Proliferation on Immobilized GAGs

B16-F10 cells were grown until confluence in 75 cm² flasks. Each flask was washed with 20 ml PBS, and treated with 3 ml trypsin-EDTA at 37° C. for 3-5 minutes, until cells detached. Cells were centrifuged for 3 minutes at 195×g. The supernatant was aspirated, and the cells were resuspended in 10 ml media. The cell density was measured using an electronic cell counter, and the suspension was diluted to 1×10⁶ or 1×10⁷ cells/ml in FBS-deficient media. Surfaces on silicone dioxide were placed on 100 mm dishes, washed twice and incubated for two hours under UV light in PBS supplemented with 100 μg/ml penicillin and 100 U/ml streptomycin. The antibiotic-treated PBS was removed, and a quantity of 130 μl cell suspension (sufficient to create a fluid film across the entirety of the GAG surface) was added to each GAG surface. To quantify cell adhesion, cells were incubated on surfaces for 2 hours, and surfaces were washed with PBS. This time point had been confirmed to be sufficient to obtain maximal adhesion of this cell type to cell culture plates. Cells attached to surfaces were quantified using an electronic cell counter after treatment with 1 ml trypsin-EDTA sufficient to detach the cells (but not to lyse them, as confirmed by light microscopy). Cell number was quantified by an electronic cell counter.

To determine the effect of various GAG surfaces on cell proliferation, cells were plated and allowed to grow for 2 hours under UV light in PBS supplemented with 100 gg/ml and 100 U/ml streptomycin. 130 μl of a 1×10⁶ or 1×10⁷ cells/ml FBS-deficient media cell suspension were added to surfaces, which were incubated for 2 hours. After 2 hours, surfaces were extensively washed with PBS to remove any cells that did not adhere. The surfaces in 100 mm dishes were supplemented with 10 ml PBS-deficient media and incubated for an additional 22, 46, 70 or 94 hours at 37° C. At the appropriate endpoint, surfaces were trypsin-treated for 20 minutes, and whole cell number was determined with an electronic cell counter. Growth was determined as the percent increase in whole cell number at the endpoint compared to the number of adhered cells.

Immunohistochemistry

B16F10 cells were added to GAG or control surfaces as described. Surfaces were washed twice with PBS after 2 hours to remove cells that did not adhere. Cells were grown on surfaces for an additional 22 hours. Cells were washed with PBS and fixed for 10 minutes in 3.7% formalin. Cells were treated with 0.1% Triton X-100 for 5 minutes and preincubated in 1% bovine serum albumin in PBS for 30 minutes.

Rabbit anti-FAK (Upstate Group, Charlottesville, Va.) and rat anti-CD44 (United States Biological, Swampscott, Mass.) were added to cells at a 1:100 dilution and incubated for 4 hours. Cells were subsequently treated with Texas red-labeled goat anti-rat secondary antibody (Molecular Probes, Eugene, Oreg.) and FITC-labeled chicken anti-goat secondary antibody (Molecular Probes) and incubated 1 hour. Cells were then treated with 4′-6-diamidino-2-phenylindole (DAPI; Molecular Probes) for 5 minutes at room temperature. Alternatively, goat polyclonal antibodies to P1 integrin (Santa Cruz Biotechnology, Santa Cruz, Calif.) were added at a 1:100 dilution and incubated 4 hours. FITC-labeled chicken anti-goat secondary antibody (Molecular Probes) and Texas red-labeled phalloidin (Molecular Probes) were added and incubated 1 hour. DAPI was then added for 5 minutes at room temperature.

Staining was then visualized by fluorescence microscopy. Controls of no antibody, primary antibody only and secondary antibody only were performed. For both staining sets, fluorescent optical images were obtained using an inverted microscope (Axiovert 200, Carl Zeiss AG) and acquired with Openlab 3.1.5 software (Improvision, Lexington, Mass.). Images were processed using Adobe Illustrator 10.0 (Adobe Systems Incorporated, San Jose, Calif.). Quantification was performed using Scion Image viewer by quantifying signal intensity for each marker and normalizing based on the number of cells in the field.

Statistical Analysis

Results are expressed as mean±standard deviation. The Student's t-test was used for statistical analysis. A p value of <0.05 was considered statistically significant.

Results

GAGs can be Immobilized to Form Stable Chemisorbed Surfaces

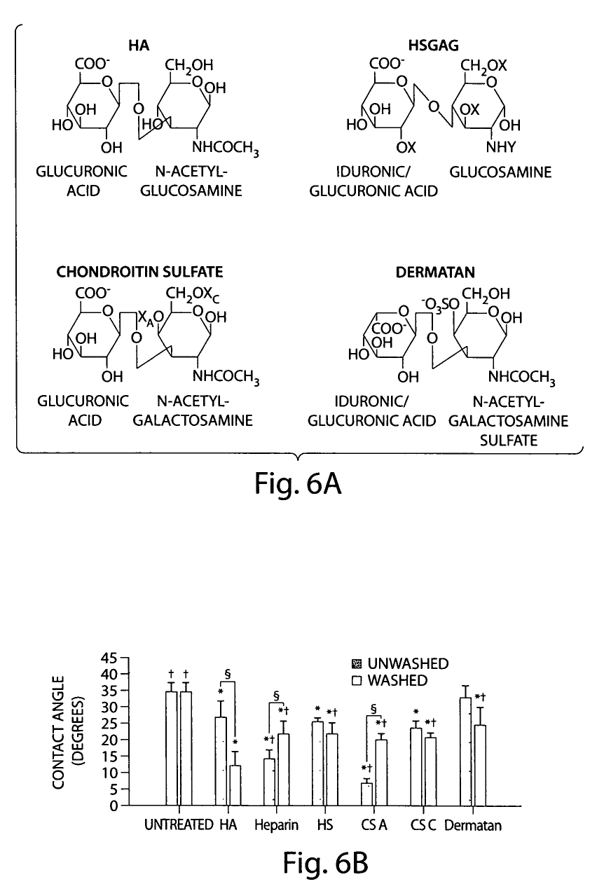

HA is composed of a well-defined disaccharide unit (FIG. 6A) without sites for variation. Other GAGs, such as HSGAGs and CSGAGs have structurally similar disaccharide units that exhibit well-defined differences (FIG. 6A). Furthermore, HSGAGs and CSGAGs have sites of intrinsic variation. In order to explore whether surfaces with variable biological activities could be produced, it was examined if GAGs in addition to HA could be used to form stable, chemisorbed surfaces.

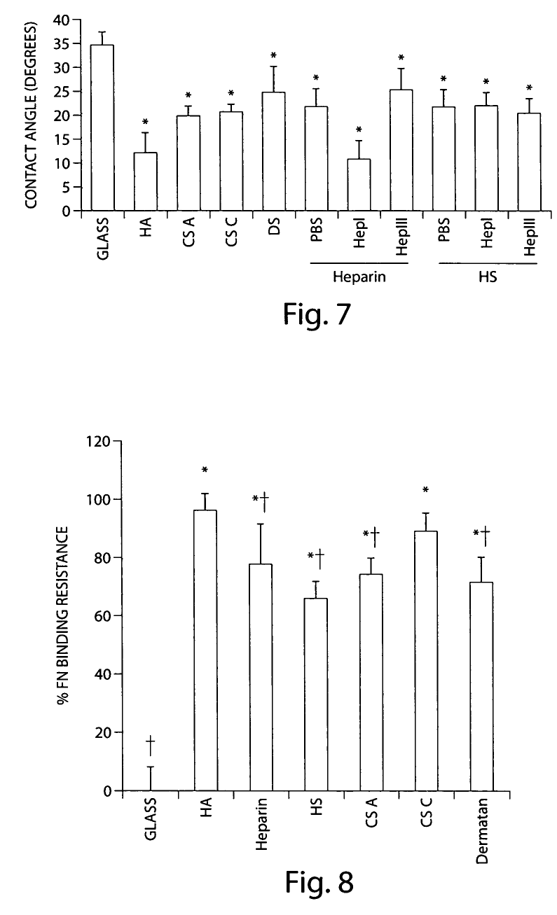

GAG surfaces were produced with HA, heparin, HS, CS A, CS C and DS (also known as CS B) as well as heparin and heparan sulfate pretreated with hepi or hep III on silicon dioxide, glass or polystyrene substrates. The successful formation of surfaces with the various GAGs was first examined on silicon dioxide by measuring the contact angle of water (FIGS. 6B and FIG. 7). The treatment of silicon dioxide wafers with each of HA (p<2×10⁻⁶), heparin (p<5×10⁻⁵), HS (p<0.001), CS A (p<2×10⁻⁵) and CS C (p<0.0001), significantly altered the contact angle of water, although treatment with DS (p>0.45) did not. After washing, the contact angles for HA (p<8×10⁻⁶), heparin (p<0.003), HS (p<0.002), CS A (p<0.0003), CS C (p<0.0005) and DS (p<0.03) were distinct from untreated silicon dioxide. The changes in contact angle suggest the presence of a hydrophilic GAG surface. Notably, all other GAGs elicited significantly different contact angles than HA after washing (p<0.02). On polystyrene, heparin (p<0.009), HS (p<0.002) and DS (p<0.05), but not HA, CS A or CS C, significantly altered the water contact angle.

The differences in the contact angles could be indicative of either the degree of surface modification or the inherent differences in the hydrophilicity of the GAGs tested. The formation of GAG surfaces was further verified and characterized by XPS. GAGs were deposited on silicon dioxide and XPS was performed to determine the relative atomic mass percentages. Nitrogen is absent in untreated surfaces, but present in the hexosamine group, which is present in all GAGs examined. Therefore, detectable nitrogen in surfaces confirm successful GAG deposition. Given that all GAGs examined contain one amine group per disaccharide, the atomic mass percentages of nitrogen allowed for quantities of GAGs immobilized to be estimated. Nitrogen was detectable after the deposition of each GAG both before and after washing (Table 3). The oxygen:carbon ratio was also altered compared to untreated silicon dioxide in surfaces created with each GAG.

TABLE 3
Layer-by-layer Deposition of GAGs Creates Distinct Surfaces

| | Nitrogen | Oxygen | Carbon | Oxygen:Carbon |
|---|---|---|---|---|
| Untreated | 0.00 | 92.42 | 7.58 | 12.19 |
| HA | 3.75 | 37.51 | 58.74 | 0.64 |
| Heparin | 0.16 | 89.43 | 10.41 | 8.59 |
| HS | 0.14 | 91.12 | 8.74 | 10.42 |
| CS A | 0.53 | 88.78 | 10.68 | 8.31 |
| CS C | 0.10 | 90.46 | 9.44 | 9.58 |
| Dermatan | 0.39 | 89.06 | 10.55 | 8.44 |

XPS was performed on GAG surfaces formed on silicon dioxide after washing. Untreated surfaces are silicon dioxide only. Numbers for nitrogen, oxygen and carbon refer to atomic mass percentage. Oxygen:carbon is the atomic mass percentage of oxygen divided by that of carbon.

The ability to form GAG surfaces on the hydrophilic silicon dioxide substrate was also examined by using ellipsometry to measure surface thickness. All GAGs examined produced detectable surfaces. HA surfaces were thickest as judged by ellipsometry. This result was confirmed by atomic force microscopy. Using similar analyses, all GAGs were found to also form surfaces on glass, and HA, heparin, HS and DS formed surfaces on plasma treated polystyrene.

Protein Resistance is Altered with Distinct GAG Surfaces

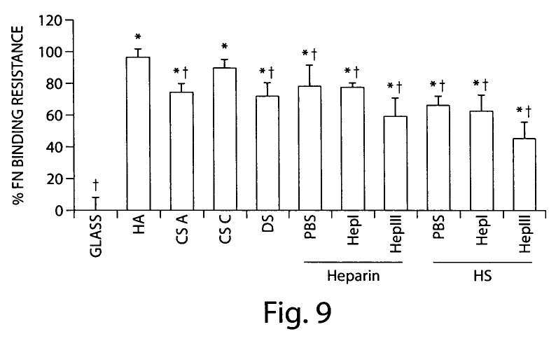

The ability of GAG surfaces to prevent protein binding was investigated. The amount of FN (FIGS. 8 and 9) and BSA that bound to GAG surfaces was compared to surfaces not treated with GAGs (the negative control) and surfaces not treated with protein (the positive control). HA inhibited 96.2±5.5% of FN binding (p<2×10⁻⁷), which was not significantly different from substrate not treated with protein (p>0.99). Heparin (77.8±13.6%; p<2×10⁻⁵), HS (66.0±5.8%; p<2×10⁻⁶), CS A (74.3±5.5%; p<9×10⁻⁷), CS C (89.2±6.1%; p<2×10⁻⁷), DS (71.5±8.8%; p<2×10⁻⁶), hepI digested heparin (77.6±2.3%; p<2×10⁻⁵), hepIII digested heparin (58.9±11.7%; p<4×10⁻⁵), hepI digested HS (62.1±9.9%; p<7×10⁻⁶) and hepIII digested HS (45.1±9.9%; p<7×10⁻⁵), each produced surfaces that significantly inhibited FN binding. Surfaces formed with heparin and CS C did not exhibit significantly more FN binding than substrate not treated with protein (p>0.09 for heparin; p>0.14 for CS C) or than HA surfaces (p>0.09 for heparin; p>0.19 for CS C). All surfaces, therefore, resisted protein binding, consistent with widespread surface formation with all GAGs examined. FN resistance additionally confirmed surface stability for at least 4 days. Similar results were observed with BSA binding.

Digestion of HSGAGs altered the ability of surfaces to resist protein adhesion compared to undigested HSGAGs. Surfaces formed with hepIII-digested heparin (p<0.02) and with hepIII-digested HS (p<0.009) allowed for significantly more protein binding than heparin and HS respectively, while treatment of either heparin or HS with hepI (p>0.27) did not alter the protein adhesive properties. Interestingly, hepIII-digested heparin yielded a surface that had similar protein binding properties as HS (p>0.70). The properties of digested HSGAGs may therefore be different from those of undigested HSGAGs, offering four additional surfaces that can be used to examine the effects on cell function.

Additionally, while XPS can only provide insight into the successful GAG deposition on a regional basis, protein adhesion can be used to observe a substantially larger field on which the surface can be created. The finding that GAGs can yield less protein binding than untreated substrates demonstrates widespread chemisorbtion of GAGs and, therefore, the formation of surfaces.

GAG Surfaces Regulate Cell Adhesive, Proliferative and Migratory Properties

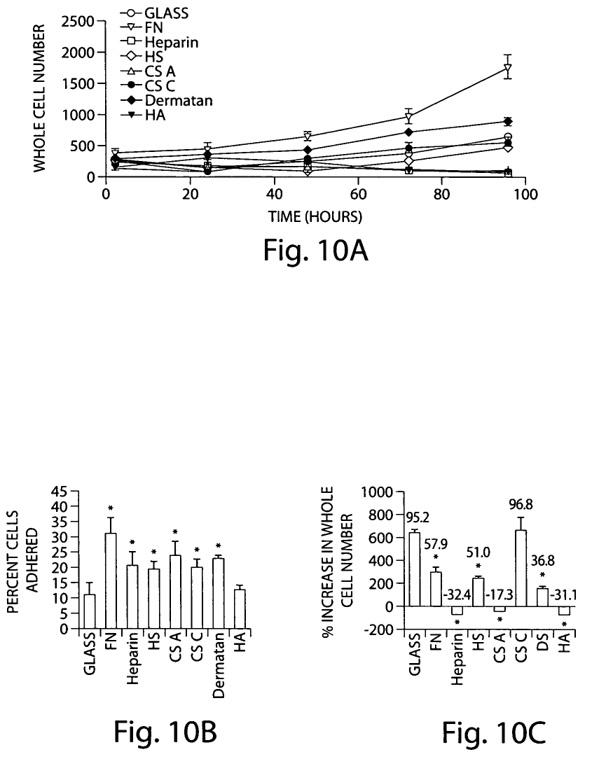

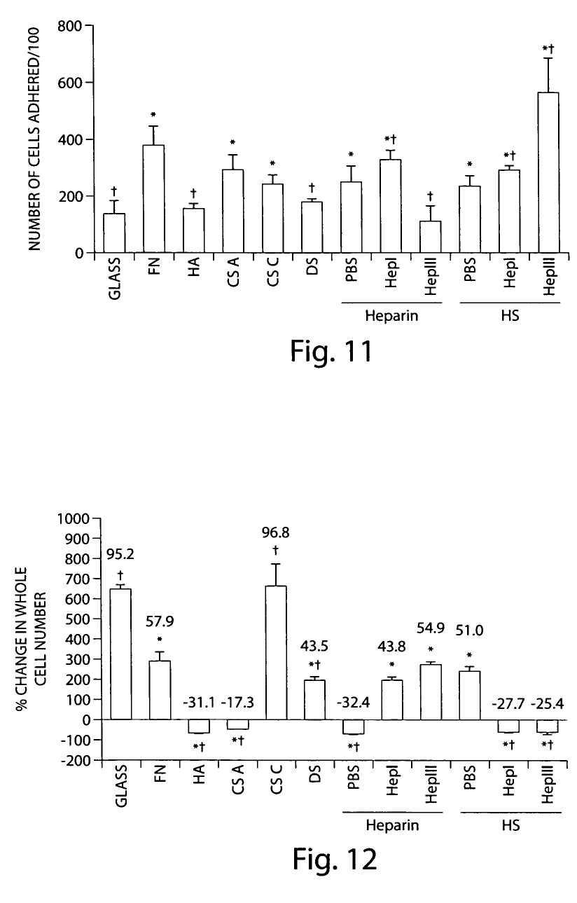

After determining that surfaces could be created with various GAGs, and that these surfaces had distinct effects on protein adhesion, how these surfaces would impact cellular behavior (e.g., cancer cell behavior) was examined. The effect on B16F10 murine melanoma cells was examined first. These cells adhered readily to plastic, even in the absence of serum. Surfaces were formed on glass with each GAG. B16F10 cells were deposited, and the number of cells adhered after two hours was determined. Only 11.1±2.9% of cells adhered to glass alone, while 30.9±5.3% adhered to glass pretreated with FN (FIG. 10A). Cells adhered to all GAG surfaces with varying degrees of efficiency (FIG. 11). HA, DS and hepIII-digested heparin surfaces resisted cell adhesion similar to glass alone (p>0.16). Heparin, HS, and CS C promoted more cell adhesion than glass alone (p<0.03), though less than FN treated glass (p<0.03). CS A, hepI-digested heparin and hepI-digested HS surfaces promoted similar cellular adhesion as FN-treated glass (p>0.07), significantly more than glass (p<0.008). HepIII-digested HS surfaces notably promoted cell adhesion more than FN-treated glass (p<0.05), with 46.1±9.7% of cells adhering. DS promoted cellular adhesion greater than glass (p<0.008) that were not significantly different from FN treated glass (p>0.05). The GAG surfaces therefore supported distinct levels of cell fimction.

After defining the adhesive properties of GAG surfaces, their effects on cell proliferation were investigated. On glass, cell number increased 643.6±23.0% over 96 hours. FN-treated glass only yielded a 293.8±42.9% increase in whole cell number. The GAG surfaces elicited distinct proliferative effects (FIGS. 10B, 10C and 12). The effects of surfaces on growth rate were consistent between the various end-points. When normalized to the number of cells adhered, surfaces formed with CS C (761.8±108.8%), DS (256.0±18.4%), hepI-digested heparin (197.2±14.1%), hepIII-digested heparin (272.2±16.4%) and HS (344.2±19.2%) promoted cell proliferation over 96 hours. Surfaces formed with HA (−67.1±5.1%), CS A (−43.4±2.5%), heparin (−69.1±5.2%), hepI-digested HS (−62.2±4.2%) and hepIII-digested HS (−58.5±12.2%) however, reduced whole cell number over four days. Surfaces with various GAGs therefore elicited distinct sets of cellular properties.

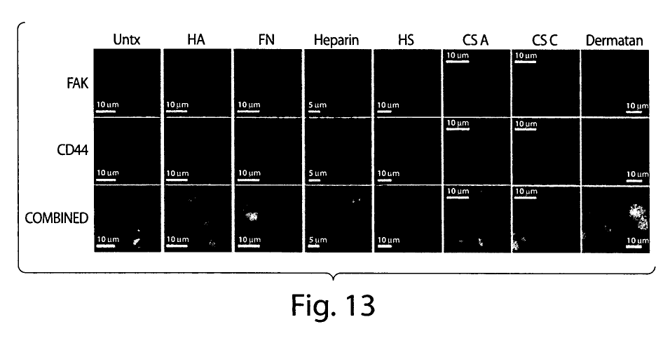

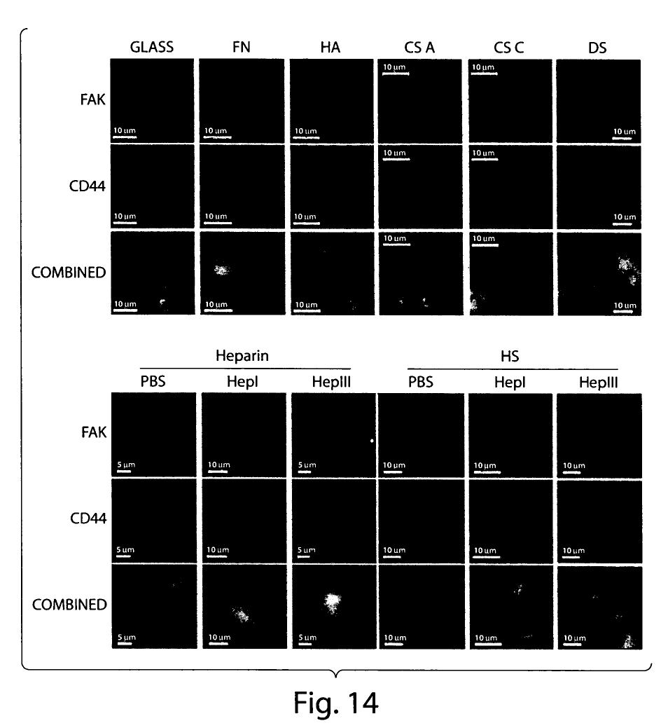

The effects on metastasis was also explored. The mechanism by which GAG surfaces influenced cellular activity was examined by immunohistochemistry. Cellular expression of β1-integrin and for f-actin was not notably altered by various GAG surfaces. The expression of FAK and CD44, however, was influenced by the surface on which cells were deposited (FIG. 13 and 14). FAK and CD44 expression were used as an in vitro surrogate for metastasis, as their expression is associated with both migration and metastasis. DS and hepIII-digested HS surfaces yielded cells with the highest expression of FAK and CD44. Intermediate levels of signaling was observed with FN, HA, CS C, hepI-digested heparin, hepIII-digested heparin and hepI-digested HS surfaces. Cells added to untreated, CS A, heparin and HS surfaces exhibited the most restricted distributions of FAK and CD44. Cellular expression of β1-integrin, which has been associated with local adhesion to a surface (Beauvais D M, Rapraeger A C. Exp Cell Res 2003; 286(2): 219-32), and for f-actin, which is associated with changes in cell-cell contacts (Dull R O, et al. Am J Physiol Lung Cell Mol Physiol 2003; 285(5): L986-95; Florian J A, et al. Circ Res 2003; 93(10): el36-42), were not altered by various GAG surfaces, verifying that the observed expression changes were marker-specific.

GAG Surfaces Elicit Biological Effects that are Distinct from those of GAGs Free in the ECM

To confirm that the cellular effects observed with GAG surfaces could be attributed to the chemisorbed nature of the GAGs rather than the GAGs alone, the ability of GAGs free in medium to alter proliferation was investigated. B16F10 cells were treated with GAGs at concentrations between 500 ng/ml and 500 μg/ml. This concentration range was selected to ensure that less, similar and greater quantities of GAGs than were found on the surfaces were examined. The total quantity of GAGs deposited on surfaces was estimated using known GAG disaccharide volumes, average disaccharide molecular weights, ellipsometry data (to provide the depth of the surfaces) and the area of slides used. Calculations using atomic mass percentage were used for confirmation. Notably, the estimates of GAG quantities for all surfaces except HA were similar enough to suggest that GAG quantity alone could not justify the distinct patterns of cellular response elicited with the different surfaces.

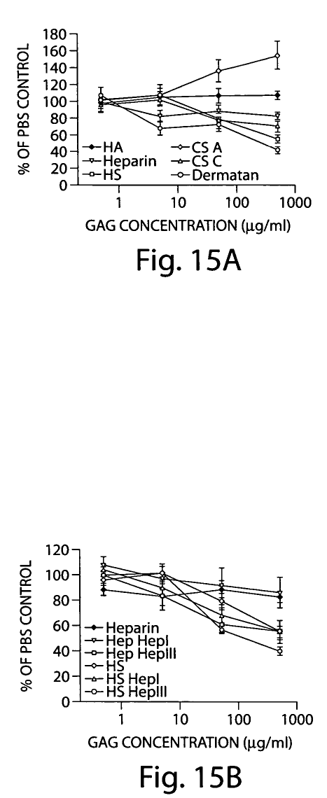

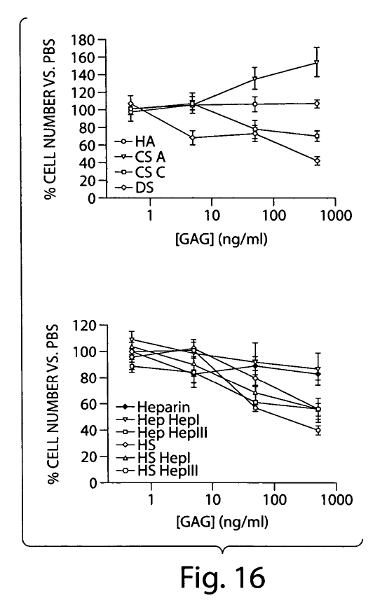

At the concentrations examined, HA (p>0.26) and heparin (p>0.14) did not alter cell proliferation (FIGS. 15A and 16). HepI-digested heparin, like untreated heparin, did not affect the proliferation of B16F10 cells (p>0.26). CS C (p<0.03), DS (p<0.002), hepIII-digested heparin (p<0.006), HS (p<0.006), hepI-digested HS (p<0.005) and hepIII-digested HS (p<0.002) surfaces inhibited B 16-F10 cell growth in a dose-dependent manner. HepIII treatment of heparin inhibited growth, reducing whole cell number by 43.7±7.4% (p<0.006). Similarly, HepI-digested HS elicited a similar growth inhibitory effect (44.9±9.0%; p<0.005) as undigested HS. The reduction in whole cell number with hepI-digested HS was not different from that of HS alone (p>0.96). HepIII treatment, however, reduced whole cell number absolutely (59.9±3.4%; p<0.002) as well as relative to undigested HS (p<0.003). At the highest concentrations, HS reduced whole cell number by 44.6±4.8% (p<0.006), CS C reduced it by 29.8±6.2% (p<0.03) and DS reduced it by 57.8±4.5% (p<0.002). CS A, however, supported cell growth, yielding a final whole cell number 154.1±16.5% (p<0.002) of that with untreated cells. Notably, the magnitude as well as the direction of the proliferative effect is starkly different, even at the highest concentrations, between cells grown on GAG surfaces and those treated with free GAGs.

To confirm that the proliferative response to immobilized GAGs was distinct from free GAGs, the percent proliferation after 72 hours compared to untreated cells was determined in both conditions, and the results for immobilized (bound) GAGs were divided by that of free GAGs. A ratio of 1.0 indicates a similar response to a given GAG presented in different manners, whereas greater or reduced ratios indicate that bound and free GAGs elicit distinct responses. Only hepIII-digested HS (1.1) had a ratio near 1.0. HA (0.26), heparin (0.32), CS A (0.20) and hepI-digested HS (0.64) free in the ECM increased whole cell number relative to the equivalent GAG surfaces. Meanwhile, surfaces produced with CS C (1.8), DS (1.9), hepI-digested heparin (1.5), hepIII-digested heparin (1.3) and HS (1.2) promoted an increased whole cell number relative to the equivalent free GAGs. The cellular effects observed with GAG surfaces are, therefore, novel and cannot be recapitulated by GAGs free in solution.

Hydrogen bonds are formed between the GAG and the substrate when GAGs are chemisorbed to produce surfaces. As a result, both the mobility of the GAGs and the potential conformations the GAGs can assume are likely reduced. The appropriate three-dimensional structures and spatial orientations of GAGs are important for functional interactions with proteins (Raman R, et al. Proc Natl Acad Sci U S A 2003; 100(5): 2357-62; Mulloy B, Forster M J. Glycobiology 200010(11)1147-56.) It is, therefore, reasonable that the ability of GAGs to alter cell function is changed when they are immobilized to produce surfaces.

Digested HSGA Gs Form Surfaces that Define Biological Function

The structural variety of the HSGAGs, heparin and HS, is much greater than that of HA or of the CSGAGs examined. Furthermore, digestion of HSGAGs can alter their biological function³. It was, therefore, examined if digested HSGAGs could be used to form surfaces similar to undigested heparin and HS, and if so, whether these surfaces could influence protein adhesion as well as cellular adhesion and proliferation. Heparin and HS were digested with hepI or hepIII for thirty minutes. The extent of digestion was measured and confirmed by UV spectroscopy at 232 nm. The degree of enzymatic cleavage was such that biological functions were evident though potentially distinct from the undigested HSGAG⁴.

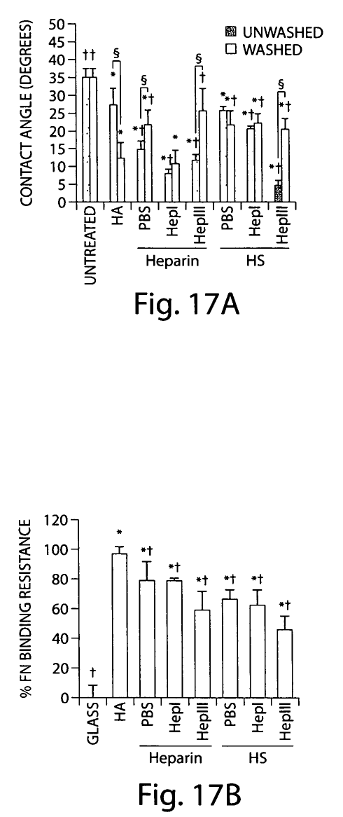

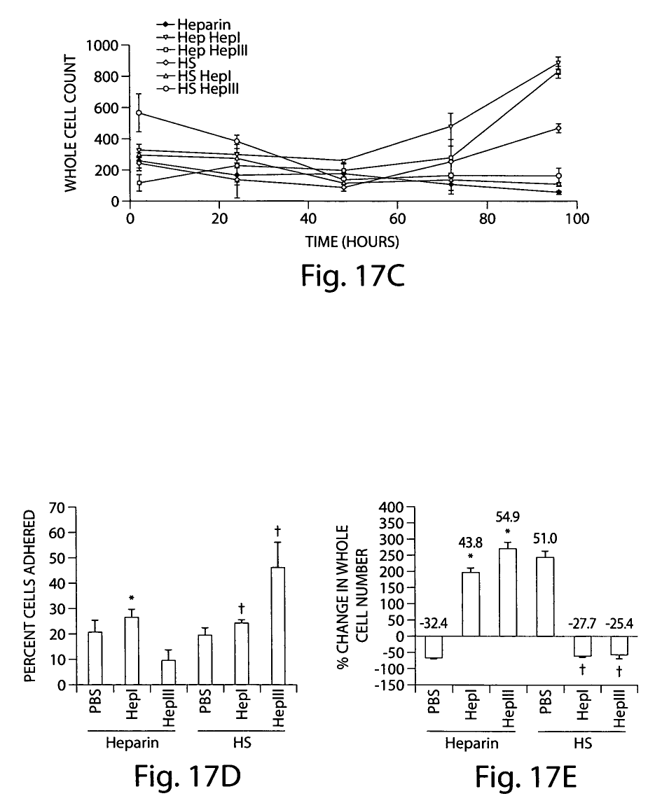

Heparin and HS, each treated with PBS, hepI, and hepIII, were deposited on glass, and the presence of surfaces was assessed. The formation of surfaces with all six HSGAGs was validated using the contact angle of water (FIG. 17A), XPS and ellipsometry. After washing, hepI digested heparin formed surfaces with distinct contact angles compared with undigested heparin (p<0.005), while hepIII digested heparin did not (p>0.37). Surfaces produced after the enzymatic treatment of HS with hepI (p>0.92) or hepIII (p>0.61) did not significantly alter the contact angle of water compared to HS.

Since surfaces could be produced with HSGAGs that were mostly similar in terms of physiochemical properties to undigested HSGAGs, their biological properties were next examined. Digestion of HSGAGs did alter the ability of surfaces to resist protein adhesion (FIG. 17B). Surfaces formed with hepIII-digested heparin (p<0.02) and with hepIII-digested HS (p<0.009) allowed for significantly more protein binding than heparin and HS respectively, while treatment with hepI (p>0.27) did not alter the protein adhesive properties. Notably, hepIII digestion of heparin yielded a surface that had similar protein binding properties as HS (p>0.70).

The similarities in structure of digested HSGAG surfaces but difference in protein resistance led us to examine the effect on cell adhesion and proliferation (FIG. 17C). Surfaces with digested HSGAGs had cell binding properties that were distinct from those of undigested HSGAGs (FIG. 17D). HepI-digested heparin was not different from undigested heparin (p>0.06). HepIII-digested heparin allowed for only 9.1±4.4% cell adhesion, which was significantly less than undigested heparin (p<0.02), and similar to glass alone (p>0.51). Surfaces formed with hepI-digested HS (23.8±1.3%; p<0.04) and with hepIII-digested HS (46.1±9.7%; p<0.01) allowed for significantly more cell adhesion than HS alone. Surfaces formed with hepIII-digested HS allowed for more cell attachment than FN (p<0.05). Digestion of HSGAGs also alters the surface properties in terms of cell proliferation (FIG. 17E). Heparin surfaces inhibited cell growth, while hepI-digested heparin surfaces (197.2±14.1%) and hepIII-digested heparin surfaces (272.2±16.4%) both supported cell growth. Conversely, HS surfaces supported cell growth, while hepI-digested HS surfaces (−62.2±4.2%) and hepIII-digested HS surfaces (−58.5±12.2%) both prevented cell growth.

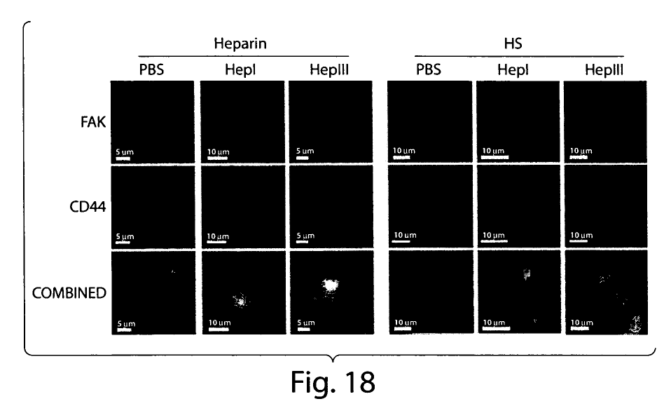

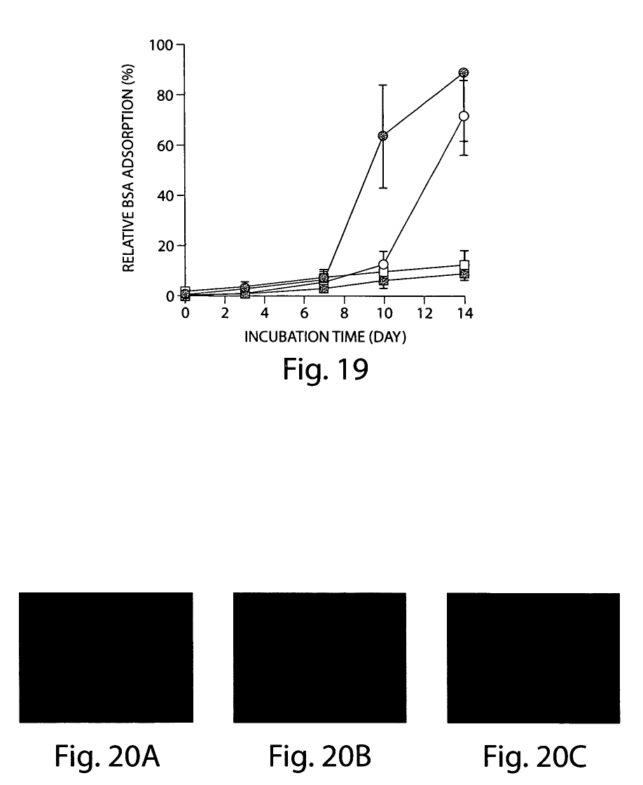

The cellular effects of digested HSGAG surfaces were further investigated by immunohistochemistry. Similar to surfaces formed with undigested GAGs, cellular expression of β1-integrin and for f-actin was not substantially altered by the surfaces formed with digested HSGAGs. FAK and CD44 expression was modulated by the digested HSGAG surfaces (FIG. 18). HepI-digested heparin and hepIII-digested heparin elicited more widespread expression of both proteins within cells relative to undigested heparin. Furthermore, hepI-digested HS reduced FAK and CD44 expression compared to undigested HS, while hepIIi-digested HS enhanced them.

It follows, therefore, that surfaces can be formed on a hydrophilic substrate, such as a silicon dioxide substrate, with one or more of the GAGs examined. In addition, some GAGs enabled surface formation on the hydrophobic polystyrene substrate. Therefore, biologically active surfaces can be formed on hydrophobic substrates as well.

Selected GAG Surfaces Have Potent Anti-cancer Activities

The ability of GAG surfaces to regulate cancer cells has been explored. Ideally, although not required, such surfaces would promote cell adhesion, but inhibit cell growth and metastasis. The specific responses of the various GAG surfaces are summarized in Table 4. In particular, it was noted that two GAG surfaces, hepIII-digested HS and heparin, had interesting and promising properties.

TABLE 4
GAG Surfaces Regulate B16-F10 Cell Activities in Distinct Manners

| | | Cell | Cell | FAK/CD44 |
|---|---|---|---|---|
| GAGs | | Adhesion | Proliferation | Expression |
| HA | | + | − | ++ |
| Heparin | PBS | ++ | − | + |
| | HepI | ++ | + | ++ |
| | HepIII | + | + | ++ |
| HS | PBS | ++ | + | + |
| | HepI | ++ | − | ++ |
| | HepIII | +++ | − | +++ |

| | | | |
|---|---|---|---|
| CS A | ++ | − | + |
| CS C | ++ | ++ | ++ |
| DS | + | + | +++ |

Each of the biological measures was stratified into three levels of responses.
Cell adhesion and FAK/CD44 expression are described as low (+), middle (++) or high (+++).
Proliferation is described as inhibited (−), promoted (+) or strongly promoted (++).

HepIII-digested HS surfaces best promoted cell adhesion and prevented proliferation. Whole cell number was reduced by 58.5±12.2% compared to the number of cells adhered over four days. B16-F10 cells added to hepIII-digested HS surfaces, however, exhibited high levels of FAK and CD44 expression, suggesting that migratory and metastatic activity may not be inhibited, and perhaps promoted. Heparin, on the other hand, elicited only moderate cell adhesion, but the greatest growth inhibitory effect, reducing whole cell number by 69.1±5.2%, and perhaps the most restricted expression pattern of FAK and CD44. Each of these surfaces has strong properties suggesting potential utility. Surfaces could also potentially be created with multiple GAGs to elicit desired responses.

Of note, the data presented also serve to screen the various GAG surfaces for other potential applications (e.g., to prevent biomaterial fouling, low protein binding, cell adhesion and cell growth, for example.) These properties are offered by, for example, HA surfaces. For a potential bioreactor system to remove metastatic cells from the blood or other bodily fluids but still enable study, ideal properties would be strong cell adhesion and cell growth, a combination of properties that could be achieved, for example, with CS C surfaces.

It has been demonstrated that GAG surfaces can regulate cancer cell activity. Comparing the effects on malignant and non-malignant cells can further establish the therapeutic value of GAG surfaces. Provided herein is a framework in which the cellular response to specific GAG surfaces can be efficiently examined. This work can be extended for the development of a biomaterial for therapeutic use to prevent cancer recurrence (e.g., after surgery).

### References for Example 2

1. Mason, M., Vercruysse, K. P., Kirker, K. R., Frisch, R., Marecak, D. M., Prestwich, G. D., and Pitt, W. G. (2000). Attachment of hyaluronic acid to polypropylene, polystyrene, and polytetrafluoroethylene. Biomaterials 21, 31-36.
3. Thierry, B., Winnik, F. M., Merhi, Y., and Tabrizian, M. (2003). Nanocoatings onto arteries via layer-by-layer deposition: toward the in vivo repair of damaged blood vessels. J Am Chem Soc 125, 7494-7495.
5. Liu, D., Shriver, Z., Venkataraman, G., El Shabrawi, Y., and Sasisekharan, R. (2002). Tumor cell surface heparan sulfate as cryptic promoters or inhibitors of tumor growth and metastasis. Proc Natl Acad Sci U S A 99, 568-573.
6. Berry, D., Kwan, C. P., Shriver, Z., Venkataraman, G., and Sasisekharan, R. (2001). Distinct heparan sulfate glycosaminoglycans are responsible for mediating Fibroblast Growth Factor-2 biological activity though different Fibroblast Growth Factor Receptors. Faseb J 15, 1422-1424.
7. Natke, B., Venkataraman, G., Nugent, M. A., and Sasisekharan, R. (2000). Heparinase treatment of bovine smooth muscle cells inhibits fibroblast growth factor-2 binding to fibroblast growth factor receptor but not FGF-2 mediated cellular proliferation. Angiogenesis 3, 249-257.
8. Berry, D., Shriver, Z., Natke, B., Kwan, C. P., Venkataraman, G., and Sasisekharan, R. (2003). Heparan sulphate glycosaminoglycans derived from endothelial cells and smooth muscle cells differentially modulate fibroblast growth factor-2 biological activity through fibroblast growth factor receptor-i. Biochem J 373, 241-249.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

We claim:

1. A composition, comprising:
a glycosaminoglycan immobilized on a substrate via hydrogen bonding, wherein the glycosaminoglycan is not hyaluronic acid.

2. The composition of claim 1, wherein the substrate is hydrophilic or hydrophobic.

3. The composition of claim 1, wherein the substrate is a hydrophobic substrate modified to contain one or more hydrophilic groups.

4. The composition of claim 3, wherein the one or more hydrophilic groups comprise a silanol, carboxylic acid or hydroxyl group or a combination thereof.

5. The composition of claim 1, wherein the glycosaminoglycan is a heparin/heparan sulfate-like glycosaminoglycan (HSGAG), a chondroitin sulfate glycosaminoglycan (CSGAG) or keratan sulfate.

6-31. (canceled)

32. A composition, comprising:
a digested glycosaminoglycan immobilized on a substrate via hydrogen bonding.

33-46. (canceled)

47. A composition, comprising:
at least two different glycosaminoglycans immobilized on a substrate, wherein at least one glycosaminoglycan is immobilized to the substrate independently from the other glycosaminoglycan.

48-72. (canceled)

73. A composition, comprising:
one or more glycosaminoglycans immobilized on a substrate, wherein the substrate comprises polystyrene, an erethylene-benzene-containing polymer or polyvinylidene chloride.

74-79. (canceled)

80. A food storage device, comprising:
one or more glycosaminoglycans immobilized on a food storage device.

81-138. (canceled)

139. A method of treating a subject, comprising:
administering a medical device to the subject and administering to the subject one or more glycosaminoglycans in an amount such that the one or more glycosaminoglycans become immobilized on the medical device.

140-145. (canceled)

146. A method of screening a cell or subcellular preparation, comprising:
contacting the composition of claim 1 with a cell or subcellular preparation, and identifying a response.

147-154. (canceled)

155. A method of determining a cellular response, comprising:
contacting the composition of claim 1 with a cell preparation, and measuring a marker for the cellular response.

156-159. (canceled)

160. A method for promoting the adhesion of proteins or cells in a subject, comprising:
providing the composition of claim 1 to a subject, wherein cells or proteins come in contact with the composition, and wherein adhesion of proteins or cells is promoted.

161. A method for promoting the adhesion of proteins or cells in vitro, comprising:
contacting a sample that contains cells or proteins with the composition of claim 1, and wherein adhesion of proteins or cells is promoted.

162. A method for inhibiting the adhesion of proteins or cells in a subject, comprising:
providing the composition of claim 1 to a subject, wherein cells or proteins come in contact with the composition, and wherein adhesion of proteins or cells is inhibited.

163. A method for resisting the adhesion of proteins or cells in vitro, comprising:
contacting a sample that contains cells or proteins with the composition of claim 1, and wherein adhesion of proteins or cells is inhibited.

164. A method for promoting the proliferation of cells in a subject, comprising:
providing the composition of claim 1 to a subject, wherein cells come in contact with the composition, and wherein the proliferation of cells is promoted.

165. A method for promoting the proliferation of cells in vitro, comprising:
contacting a sample that contains cells with the composition of claim 1, and wherein the proliferation of cells is promoted.

166. A method for inhibiting the proliferation of cells in a subject, comprising:
providing the composition of claim 1 to a subject, wherein cells come in contact with the composition, and wherein the proliferation of cells is inhibited.

167. A method for inhibiting the proliferation of cells in vitro, comprising:
contacting a sample that contains cells with the composition of claim 1, and wherein the proliferation of cells is inhibited.

168. A method for inhibiting bacterial or viral adhesion in a subject, comprising:
providing the composition of claim 1 to a subject, wherein bacteria or viruses come in contact with the composition, and wherein bacterial or viral adhesion is inhibited.

169. A method for inhibiting bacterial or viral adhesion in vitro, comprising:
contacting a sample that contains bacteria or viruses with the composition of claim 1, and wherein bacterial or viral adhesion is inhibited.

170. A method for promoting bacterial or viral adhesion in vitro, comprising:
contacting a sample that contains bacteria or viruses with the composition of claim 1, and wherein bacterial or viral adhesion is promoted.

171. A method for preventing food contamination or spoilage, comprising:
contacting a food with the composition of claim 73, and whereby food contamination or spoilage is prevented.

172-174. (canceled)

175. A method for preventing food contamination or spoilage, comprising:
contacting a food with the food storage device of claim 80, and whereby food contamination or spoilage is prevented.

176-180. (canceled)
